(19) United States
(12) Patent Application Publication
(10) Pub. No.: US 2023/0187020 A1
(43) Pub. Date: Jun. 15, 2023

(54) SYSTEMS AND METHODS FOR ITERATIVE AND SCALABLE POPULATION-SCALE VARIANT ANALYSIS

(71) Applicants: Illumina Software, Inc., San Diego, CA (US); Illumina Cambridge Limited, Cambridge (GB)

(72) Inventors: Zhuoyi Huang, Chula Vista, CA (US); Jacobus De Beer, San Diego, CA (US); Ole Benjamin Schulz-Trieglaff, Cambridge (GB); Adam Birnbaum, La Jolla, CA (US); Bernardo Ochoa Montaño, Cambridge (GB)

(73) Assignees: Illumina Software, Inc., San Diego, CA (US); Illumina Cambridge Limited, Cambridge (GB)

(21) Appl. No.: 18/082,229

(22) Filed: Dec. 15, 2022

Related U.S. Application Data
(60) Provisional application No. 63/361,386, filed on Dec. 15, 2021.
(60) Provisional application No. 63/326,227, filed on Mar. 31, 2022.

(51) Int. Cl. G16B 20/00; G16B 30/00; G16B 40/20

(52) U.S. Cl. CPC G16B 20/00; G16B 30/00; G16B 40/20

(57) ABSTRACT

An iterative process may be implemented for incrementally aggregating available batches of sample data with previously available batches to perform sequencing analysis. Genomic variant call files associated with one or more samples may be received in batches from sequencing devices and aggregated for performing sequencing analysis. The aggregated genomic variant call files may be used to generate cohort files and census files that comprise summary information related to the genomic variant call files in each batch. The census data in census files may be aggregated into a global census file that includes summary genome variant data. Multi-sample variant call files may be generated based on the global census file, cohort files, and census files. The genomic variant call files may be processed using parallel processing at multiple compute nodes. The files may be further compressed and overlapping data may be efficiently stored in buffer positions.

### CROSS-REFERENCE TO RELATED APPLICATIONS

This application claims priority to U.S. provisional patent application No. 63/361,386, filed Dec. 15, 2021, and U.S. provisional patent application No. 63/326,227, filed Mar. 31, 2022, which are incorporated herein by reference in their entirety.

### BACKGROUND

Population-scale genomics experiments can include aggregating and/or merging data associated with variants from a large number (e.g., hundreds of thousands) of samples. Existing sequencing analysis may focus on per sample analysis. As sequencing throughput continues to increase, timely delivery of population-scale variant analysis results has become increasingly desired. Moreover, in existing sequencing analyses the sequencing of samples may be performed such that some data may be unavailable at any given point in time.

The existing sequencing analyses may use a genome variant call format (gVCF) file. The gVCF file stores sequencing information for both variant and non-variant positions. The gVCF file may enable representation of genotype, annotation, and other information across all sites in the genome. A gVCF genotyper may be a population-based analysis tool that jointly analyzes variants from unrelated individuals.

### SUMMARY

Systems, methods, and apparatus are described herein for enabling an iterative process of incrementally aggregating available batches of sample data with previously available batches. The one or more computing devices may be configured to receive one or more genomic variant call files associated with one or more samples. An example of the genomic variant call files may be a genome variant call format (gVCF) file. The data in the genomic variant call files may include a list of variants and genomic blocks. The genomic variant call files may be received in batches of samples conducted by sequencing devices at different sites.

A cohort file and a census file may be generated for each batch of samples. The cohort files and the census files may comprise a subset of fields in the genomic variant call file and may include summary information of the batch of samples for the subset of fields. The census data in multiple census files generated from different batches of samples may be aggregated into a global census file.

Multi-sample variant call files may be generated based on the global census file, one or more cohort files, and one or more census files. The multi-sample variant call file may be stored in memory for performing sequencing analysis on the data in the file. For example, one or more computing devices may be implemented to perform a genome-wide sequencing analysis using one or more multi-sample variant call files.

The genomic variant call files may be processed using parallel processing at multiple compute nodes, as described herein. Each batch of genomic variant call files may be split into shards of equal size for enabling parallel processing of the genomic variant call files. Each shard may be processed using one of a plurality of computation nodes. The parallel processing may be implemented using multithreading by regions of sequence data. At least two compute nodes may be configured to perform at least two levels of parallelization for processing, aggregating, and/or generating data for a corresponding region of the sequence data. Each compute node may be configured to process a specific region. Each core may have specific threading. A variable number of software threads may be implemented. One or more threads may be implemented for each CPU core. For example, a single thread may be implemented by each CPU core. The number of threads implemented by each CPU core may be changed in response to user input.

One or more computing devices may be implemented as described herein to perform allele ordering and genotype reindexing. Each genomic variant call file may be associated with a respective sample of a plurality of samples that includes reference alternate genotype (RAGT) statistics. Using the RAGT statistics, a plurality of reference alleles and a plurality of alternate alleles may be identified that are associated with the samples in the genomic variant call files. The instances of each of the plurality of reference alleles and each of the plurality of alternate alleles may be summed for normalization to determine the number of unique alleles. A normalized reference allele may be selected from the plurality of reference alleles. The longest reference allele may be selected as the normalized reference allele. The other reference alleles of the plurality of reference alleles may be normalized by extending to correspond to the normalized reference allele. The plurality of alternate alleles may be normalized by extending each alternate allele the same amount that the respective corresponding reference allele was extended. A multi-sample variant call file may be generated using the normalized reference alleles and the normalized alternate alleles.

One or more computing devices may be implemented to perform compression on data stored in cohort files and/or census files. A field of the cohort file or the census file being compressed may be identified. In one example, a plurality of reference alleles and a plurality of alternate alleles associated with the plurality of samples may be stored in an RAGT field that may be identified for compression. The one or more computing devices may be configured to determine which of the plurality of samples have common reference and alternate alleles. The samples may be distributed into allele groups. Each allele group may comprise one or more samples with common reference and alternate alleles. The one or more computing devices may be configured to select a binary value length based on the number of allele groups. The binary value length may be the shortest binary value length that can be used to uniquely identify each of the allele groups. The one or more computing devices may be configured to assign, using the determined binary value length, a unique binary value to each of the allele groups. The unique binary value for each of the allele groups may be stored in a bitmap that is used to encode the plurality of reference alleles and the plurality of alternate alleles in a bit array.

Genome variant data in cohort files and census files may be aggregated in an output buffer comprising a fixed number of buffer positions, as described herein. For example, one or more computing devices may be configured to receive records of genome variant data and determine whether the genome variant data for a received record overlaps with one or more other previously stored records in the output buffer. When the genome variant data for the received record fails to overlap with a previously stored record in the buffer, the genome variant data for the received record may be stored in one or more buffer positions in the buffer. When the genome variant data for the received record overlaps with the genome variant data of another record, the buffer position comprising the overlapping portion of the records may be updated to include the genome variant data for the received record. Any non-overlapping portions of the previously stored record may be copied and stored in sequential buffer positions with the overlapping portion of the records. Any non-overlapping portions of the record being received may be added to sequential buffer positions with the overlapping potion of the records.

### BRIEF DESCRIPTION OF DRAWINGS

FIG. 1 illustrates a schematic diagram of a system environment (or “environment”) in which an iterative genome variant call format (gVCF) genotyper may operate.

FIG. 2 illustrates a block diagram of an example computing device.

FIG. 3 is an illustration depicting an example process that may be implemented by one or more computing devices to perform iterative gVCF genotyping.

FIG. 4 is a flowchart of an example method for processing batches of sample data from a sequencing device for generating one or more multi-sample variant call format (msVCF) files.

FIG. 5A is an illustration depicting an example process performed by an iterative gVCF genotyper.

FIG. 5B is an illustration depicting another example process performed by an iterative gVCF genotyper.

FIG. 6 is a graphical representation of an example cohort record generated for a batch of samples.

FIG. 7 is an illustration depicting an example iterative process that may be implemented by one or more computing devices to generate multi-sample variant call files after receipt of gVCF files associated with a batch.

FIG. 8 is an illustration depicting an example process that may be implemented by one or more computing devices to incorporate parallel processing in the variant analysis.

FIG. 9 is an illustration depicting an example process incorporating parallel processing across batches.

FIG. 10 is an illustration depicting an example Genome Data Operator design.

FIG. 11 is an illustration depicting an example process that may be implemented by one or more computing devices for reindexing a genotype for an msVCF file.

FIG. 12A is an illustration depicting an example process that may be implemented by one or more computing devices to encode cohort data.

FIG. 12B is an illustration depicting an example process that may be implemented by one or more computing devices to encode census data.

FIG. 13 is a flow diagram depicting an example method that may be implemented by one or more computing devices to perform compression of cohort and/or census data.

FIG. 14 is an illustration depicting an example process that may be implemented by one or more computing devices for data serialization and compression.

FIG. 15 is an illustration depicting another example process that may be implemented by one or more computing devices for data serialization and compression.

FIG. 16 is an illustration depicting an example process that may be implemented by one or more computing devices for record adding, copying, and/or updating data during buffer aggregation with overlapping genomic regions.

### DETAILED DESCRIPTION

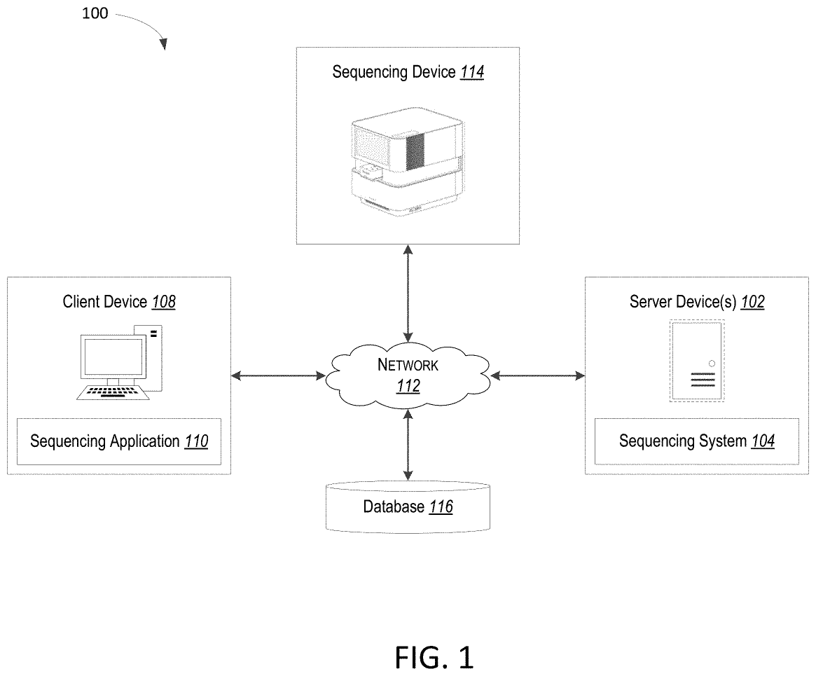

FIG. 1 illustrates a schematic diagram of a system environment (or “environment”) 100 in which an iterative genome variant call format (gVCF) genotyper may operate, as described herein. As illustrated, the environment 100 includes one or more server device(s) 102 connected to a client device 108 and a sequencing device 114 via a network 112.

As shown in FIG. 1, the server device(s) 102, the client device 108, and the sequencing device 114 may communicate with each other via the network 112. The network 112 may comprise any suitable network over which computing devices can communicate. The network 112 may include a wired and/or wireless communication network. Example wireless communication networks may be comprised of one or more types of RF communication signals using one or more wireless communication protocols, such as a cellular communication protocol, a WIFI communication protocol, and/or another wireless communication protocol. In addition, or in the alternative to communicating across the network 112, the server device(s) 102, the client device 108, and/or the sequencing device 114 may communicate may bypass the network 112 and may communicate directly with one another.

As indicated by FIG. 1, the sequencing device 114 may comprise a device for sequencing a biological sample. The biological sample may include human and non-human DNA to determine individual nucleotide bases of nucleic-acid sequences (e.g., sequencing by synthesis). The sequencing device 114 may analyze nucleic-acid segments and/or oligonucleotides extracted from samples to generate nucleotide reads and/or other data utilizing computer implemented methods and systems described herein either directly or indirectly on the sequencing device 114. More particularly, the sequencing device 114 may receive and analyze, within nucleotide-sample slides (e.g., flow cells), nucleic-acid sequences extracted from samples. The sequencing device 114 may utilize SBS to sequence nucleic-acid segments into nucleotide reads.

As further indicated by FIG. 1, the server device(s) 102 may generate, receive, analyze, store, and/or transmit digital data, such as data for determining nucleotide-base calls or sequencing nucleic-acid polymers. As shown in FIG. 1, the sequencing device 114 may send (and the server device(s) 102 may receive) call data from the sequencing device 114. The server device(s) 102 may also communicate with the client device 108. In particular, the server device(s) 102 may send data to the client device 108, including a variant call file or other information indicating nucleotide-base calls, sequencing metrics, error data, and/or other metrics associated with a nucleotide-base call, such as a call quality, a genotype, and/or a genotype quality.

The server device(s) 102 may comprise a distributed collection of servers where the server device(s) 102 include a number of server devices distributed across the network 112 and located in the same or different physical locations. Further, the server device(s) 102 may comprise a content server, an application server, a communication server, a web-hosting server, or another type of server.

As further shown in FIG. 1, the server device(s) 102 may include a sequencing system 104. The sequencing system 104 may analyze base call data, such as sequencing metrics received from the sequencing device 114, to determine nucleotide base sequences for nucleic-acid polymers. For example, the sequencing system 104 may receive raw data from the sequencing device 114 and may determine a nucleotide base sequence for a nucleic-acid segment. The sequencing system 104 may determine the sequences of nucleotide bases in DNA and/or RNA segments or oligonucleotides. In addition to processing and determining sequences for biological samples, the sequencing system 104 may generate a variant call file indicating one or more nucleotide-base calls for one or more genomic coordinates. The sequencing system 104 may comprise one or more iterative gVCF genotypers capable of performing analysis, file generation, data aggregation, compression, and/or serialization, as described herein. The iterative gVCF genotypers may also be distributed across the sequencing system 104, the sequencing application 110, and/or the database 116 to perform as described herein. A primary analysis may be defined as calling the individual nucleotide bases. A secondary analysis may be defined as alignment and/or assembly of DNA or RNA fragments (e.g., one or more nucleotide bases) by mapping reads to a reference genome. A tertiary analysis may be defined as variant identification/calling based on primary and/or secondary analysis. The primary analysis, the second analysis, and/or the tertiary analysis may be performed on or off instrument.

The client device 108 may generate, store, receive, and/or send digital data. In particular, the client device 108 may receive sequencing metrics from the sequencing device 114. Furthermore, the client device 108 may communicate with the server device(s) 102 to receive a variant call file comprising nucleotide base calls and/or other metrics, such as a call-quality, a genotype indication, and a genotype quality. The client device 108 may present or display information pertaining to the nucleotide-base call within a graphical user interface to a user associated with the client device 108. For example, the client device 108 may present a contribution-measure interface that includes a visualization or a depiction of various contribution measures associated with, or attributed to, individual sequencing metrics with respect to a particular nucleotide-base call.

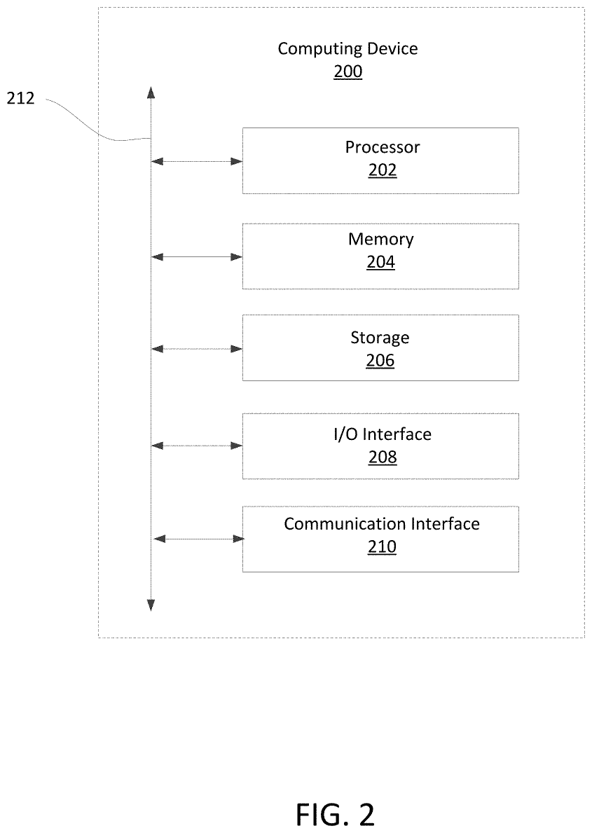

The client device 108 illustrated in FIG. 1 may comprise various types of client devices. In examples, the client device 108 may include non-mobile devices, such as desktop computers or servers, or other types of client devices. In other examples, the client device 108 may include mobile devices, such as laptops, tablets, mobile telephones, or smartphones. Additional details regarding the client device 108 are discussed below with respect to FIG. 2.

As further illustrated in FIG. 1, the client device 108 may include a sequencing application 110. The sequencing application 110 may be a web application or a native application stored and executed on the client device 108 (e.g., a mobile application, desktop application). The sequencing application 110 may include instructions that (when executed) cause the client device 108 to receive data from the sequencing device 114 and present, for display at the client device 108, data from a variant call file. Furthermore, the sequencing application 110 may instruct the client device 108 to display a visualization of contribution measures for sequencing metrics of a nucleotide-base call.

As further illustrated in FIG. 1, the environment 100 may include a database 116. The database 116 can store information such as variant call files, sample nucleotide sequences, nucleotide reads, nucleotide-base calls, and sequencing metrics. The server device(s) 102, the client device 108, and/or the sequencing device 114 may communicate with the database 116 (e.g., via the network 112) to store and/or access information, such as variant call files, sample nucleotide sequences, nucleotide reads, nucleotide-base calls, and/or sequencing metrics. The database 116 may also store one or more models, such as a call-recalibration-machine-learning model and/or a call-generation model.

The environment 100 may be included in a local network or local high-performance computing (HPC) system. For example, the iterative gVCF genotypers described herein may be executed on one or more client devices 108 (e.g., as a part of the sequencing application 110), one or more server devices 102 (e.g., as a part of the sequencing system 104), and/or one or more sequencing devices 114 in a local network or HPC system. The environment 100 may be included in a cloud computing environment comprising a plurality of server devices, such as server device(s) 102, having software and/or data distributed thereon. For example, the iterative gVCF genotypers described herein may be executed on one or more server devices 102 in a cloud computing environment. The sequencing system 104 may be implemented to execute the gVCF genotypers described herein, and may be distributed across server devices 102 having access to the database 116 via the network 112 in a cloud-based computing system.

Though FIG. 1 illustrates the components of environment 100 communicating via the network 112, it will be appreciated that the components of environment 100 may communicate directly with each other, for example, bypassing the network 112. For example, the client device 108 may communicate directly with the sequencing device 114.

FIG. 2 illustrates a block diagram of an example computing device 200. One or more computing devices such as the computing device 200 may implement one or more features of the iterative gVCF genotyper described herein and/or the sequencing system 104. One or more computing devices such as the computing device 200 may operate as a client device, server device, or a sequencing device, as described herein. As shown by FIG. 2, the computing device 200 may comprise a processor 202, a memory 204, a storage device 206, an I/O interface 208, and/or a communication interface 210, which may be communicatively coupled by way of a communication infrastructure 212. It will be appreciated that the computing device 200 may include fewer or more components than those shown in FIG. 2.

The processor 202 may include hardware for executing instructions, such as those making up a computer program. In examples, to execute instructions for dynamically modifying workflows, the processor 202 may retrieve (or fetch) the instructions from an internal register, an internal cache, the memory 204, or the storage device 206 and decode and execute the instructions. The memory 204 may be a volatile or non-volatile memory used for storing data, metadata, and programs for execution by the processor(s). The storage device 206 may include storage, such as a hard disk, flash disk drive, or other digital storage device, for storing data or instructions for performing the methods described herein. The memory 204 may have stored thereon computer-readable or machine-readable instructions for performing one or more processes or methods described herein.

The I/O interface 208 may allow a user to provide input to, receive output from, and/or otherwise transfer data to and receive data from the computing device 200. The I/O interface 208 may include a mouse, a keypad or a keyboard, a touch screen, a camera, an optical scanner, network interface, modem, other known I/O devices or a combination of such I/O interfaces. The I/O interface 208 may include one or more devices for presenting output to a user, including, but not limited to, a graphics engine, a display (e.g., a display screen), one or more output drivers (e.g., display drivers), one or more audio speakers, and one or more audio drivers. The I/O interface 208 may be configured to provide graphical data to a display for presentation to a user. The graphical data may be representative of one or more graphical user interfaces and/or any other graphical content.

The communication interface 210 may include hardware, software, or both. In any event, the communication interface 210 may provide one or more interfaces for communication (such as, for example, packet-based communication) between the computing device 200 and one or more other computing devices or networks. As an example, and not by way of limitation, the communication interface 210 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 210 may facilitate communications with various types of wired or wireless networks. The communication interface 210 may also facilitate communications using various communication protocols. The communication infrastructure 212 may also include hardware, software, or both that couples components of the computing device 200 to each other. For example, the communication interface 210 may use one or more networks and/or protocols to enable a plurality of computing devices connected by a particular infrastructure to communicate with each other to perform one or more aspects of the processes described herein. To illustrate, the sequencing process may allow a plurality of devices (e.g., a client device, sequencing device, and server device(s)) to exchange information such as sequencing data and error notifications.

The computing devices, systems, and portions thereof described herein may be implemented to assist in genome sequencing, including genomic variant calling and processing of genotyping files, as described herein. The one or more computing devices may be configured to receive one or more genomic variant call files associated with one or more samples. An example of the genomic variant call files may be a genome variant call format (gVCF) file. gVCF genotypers may be implemented by one or more computing devices to perform sequencing analysis on the genomic variant call files, as described herein. For example, a gVCF genotyper may be implemented at one or more computing devices and may receive sample data from one or more sequencing devices to generate a gVCF file. The gVCF file may be a digital file generated in a publicly available standard text format that includes a number of predefined fields of summary information related to a sample, such as genome variant data, related to the sample to which the gVCF file corresponds. The summary information in the gVCF file may include genome variant data about the variants and non-variant genomic blocks at specific genomic coordinates, including meta-information lines, a header line, and data lines where each data line contains information about a single nucleotide-base call (e.g., a single variant). The genome variant data in the gVCF file may include one or more nucleotide-base calls (e.g., variant calls) along with other information pertaining to the nucleotide-base calls (e.g., variant calls, quality, mapping alignment, and other metrics).

As gVCF genotypers may be focused on per-sample high performance sequencing analysis, the genome variant data in each gVCF file may include information related to a single sample from a single sequencing run, a sequencing cycle, or multiple sequencing runs at a sequencing device. A gVCF genotyper may take the summary information from a batch of multiple gVCF files, which each correspond to a single sample from a sequencing device, and analyze the information in an attempt to identify aggregate genome variant data and/or other genotype data from the batch of gVCF files. Due to the size of the gVCF files, the storage of the gVCF files may utilize large amounts of memory resources and analysis of the gVCF files may utilize large amounts of processing resources. In one example, the gVCF files may utilize 48 threads and/or 250 GB of memory for analysis on a local server or other computing device. In an example cloud instance (e.g., on an AWS c5d.18×large), the gVCF files may utilize 72 threads and/or 144 GB of memory for analysis. In another example of the processing power that may be utilized for analyzing a single gVCF file, the gVCF file may utilize 1 thread and/or 4 GB of memory, though additional resources may be implemented.

Additionally, it may be desirable to also analyze samples that have been taken by multiple sequencing devices across multiple sites to identify aggregate genome variant data and/or other genotype data across sequencing devices to build a dataset having a larger aggregate dataset of genome variant data and other genotype data. In fact, in some example embodiments, a gVCF genotyper feature may be used to implement Genome Analysis Toolkit (GATK) algorithms to aggregate and join genome variant data from large cohorts. Due to the size of the gVCF files, aggregating and joining genome variant data and other genotype data in batches of gVCF files from multiple sequencing devices at multiple sites may be difficult to scale (e.g., beyond several thousand samples). The batches of gVCF files from multiple sequencing devices may be difficult to store, analyze, and/or aggregate for identifying aggregate genome variant data and/or other genotype data gathered across the sequencing devices. The size of a single gVCF file may be relatively large when compared to other file types that may be used for storing and analyzing data, as the gVCF files are comprised of text fields that may occupy a greater amount of storage than other field types. For example, a gVCF file with a 30× sequencing depth may be 4 to 5 GB. As the gVCF files are aggregated for performing analysis of the data across batches or different sites from which the samples are being received, the storage and analysis of the data in the gVCF files may become increasingly difficult. Due to the size of the gVCF files, it may also be difficult to communicate batches of sample data from different sites to aggregate variant information or other summary information from sites for identifying variant information across sites.

To provide an example of the size of the standard gVCF files that are used for sequencing analysis, we describe herein the plurality of fields (e.g., which comprise example genome variant data) that are utilized in the standard format. For example, the plurality of fields in the gVCF file(s) may include a genotype (GT) field, a genotype quality (GQ) field, a minimum of genotype quality (GQX) field, a filtered base call depth (DP) field, a base calls filtered from input (DPF) field, an allelic depth (AD) field, a read depth associated with indel (DPI) field, a mapping qualities (MQ) field, a filter (FT) field, a quality (QL) field, a phred-scaled genotype likelihood (PL) field, and a reference allele, one or more alternate alleles+genotype (GT) field, a contig name (CHROM), the start and end position of the record (POS, END), the reference allele sequence (REF), and/or the sequence of one or more alternate alleles (ALT).

The GT field may be encoded as allele values separated by a delimiter (e.g., either of/or |). The allele values may be zero for the reference allele (e.g., what is in the REF field), one (1) for the first allele listed in ALT, two (2) for the second allele list in ALT, and so on. For diploid calls, allele value examples may include 0/1, 1|0, or 1/2, etc. For haploid calls, e.g., on Y, male nonpseudoautosomal X, or mitochondrion, one (e.g., only one) allele value may be given; a triploid call may be 0/0/1. If a call (e.g., allele call) cannot be made for a sample at a given locus, ‘.’ may be specified for each missing allele 5 in the GT field (for example ‘./.’ for a diploid genotype and ‘.’ for haploid genotype). The separator °/: may represent an unphased genotype. The separator °|: may represent a phased genotype. The REF field may indicate one or more reference bases (e.g., A, C, G, T, N). The ALT field may be a comma separated list of alternate non-reference alleles called on at least one of the samples.

The GQ field may indicate conditional genotype quality encoded as a phred quality −10 log 10 probability of the genotype call being wrong (e.g., the error rate of genotyping), conditioned on the site's being variant (Integer). As indicated, the GQ field may store a log transformed probability of an error that indicates whether a genotype call is correct or incorrect.

The GQX field may indicate the genotype quality assuming variant position or assuming non-variant position.

The filtered base call depth may be used for site genotyping. The DP field may be an integer value.

The AD field may indicate allelic depths for the ref and alt alleles, for example, in the order listed. For indels, this value may include reads that confidently support each allele, for example, having a posterior probability of 0.999 or higher that read contains indicated allele vs. other (e.g., all other) intersecting indel alleles.

The DPI field may be taken from a site preceding the indel.

The MQ field may indicate RMS mapping quality. The MQ field may be an integer value. The system may identify or generate mapping quality scores for nucleobase calls at genomic coordinates, where a MAPQ score represents −10 log 10 probability of the read mapping position being wrong (e.g., the error rate of read mapping position), rounded to the nearest integer. As indicated, the MAPQ score may include a log transformed probability of an error that indicates whether a read mapping position is correct or incorrect. Additionally or alternatively, the system may determine soft-clipping metrics for sample nucleic-acid sequences by, for example, determining a total number of soft-clipped nucleobases spanning a genomic coordinate. If any of the fields is missing, it is replaced with the missing value. For example, if the FORMAT is GT:GQ:DP:HQ then 0|0: . : 23: 23, 34 indicates that GQ is missing. Trailing fields can be dropped (with the exception of the GT field, which should always be present if specified in the FORMAT field). See below for additional genotype fields used to encode structural variants. Additional Genotype fields can be defined in the meta-information. However, software support for such fields is not guaranteed.

The FT field may include a sample genotype filter indicating if this genotype was “called” (e.g., similar in concept to the FILTER field). PASS may be used in the FT field to indicate that all filters have been passed. A semicolon-separated list of codes may be used in the FT field to indicate one or more filters that fail. A period ‘.’ may be used in the FT field to indicate that filters have not been applied. These FT field values may be described in the meta-information in the same way as FILTERs. The FT field may be a String with no whitespace or semicolons permitted.

The QL field may indicate a phred-scaled quality score (e.g., for the assertion made in the ALT field −10 log 10 probability of the call in ALT being wrong (e.g., the error rate of ALT)). The phred-scaled quality score may be a measure of base calling accuracy. The phred-scaled quality score may be used to assess the accuracy of a sequencing platform. The phred-scaled quality score may indicate the probability that a given base is called incorrectly by the sequencer. If the ALT field indicates ‘.’ (no variant) then the phred-scaled quality score may be calculated as −10 log 10 prob(variant), and if ALT is not ‘.’ the phred-scaled quality score may be calculated as −10 log 10 prob(no variant). If unknown, the MISSING value must be specified.

The PL field may indicate the phred-scaled genotype likelihoods rounded to the closest integer (e.g., and otherwise defined precisely as the GL field). The PL field may be an integer value.

The RAGT (reference alternate genotype) field may be a combination of “Reference allele+Alternate allele+Genotype” from one sample in one genomic region or at one genomic position. The RAGT field may be critical in calculation of allele frequency, allele count, and/or normalization of different variant alleles across large number of samples. The RAGT statistics (e.g., in the RAGT field) may indicate how many samples with what kinds of alleles are at each genomic position. The RAGT statistics (e.g., in the RAGT field) may indicate whether a particular region in the genome is difficult to sequence and/or genotype. The RAGT values may include key information on allele frequency and variants. The RAGT may hash the reference allele, the alternate allele, and the genotype of one sample at one genomic position or registration into one key. The key may be population-genotype-specific.

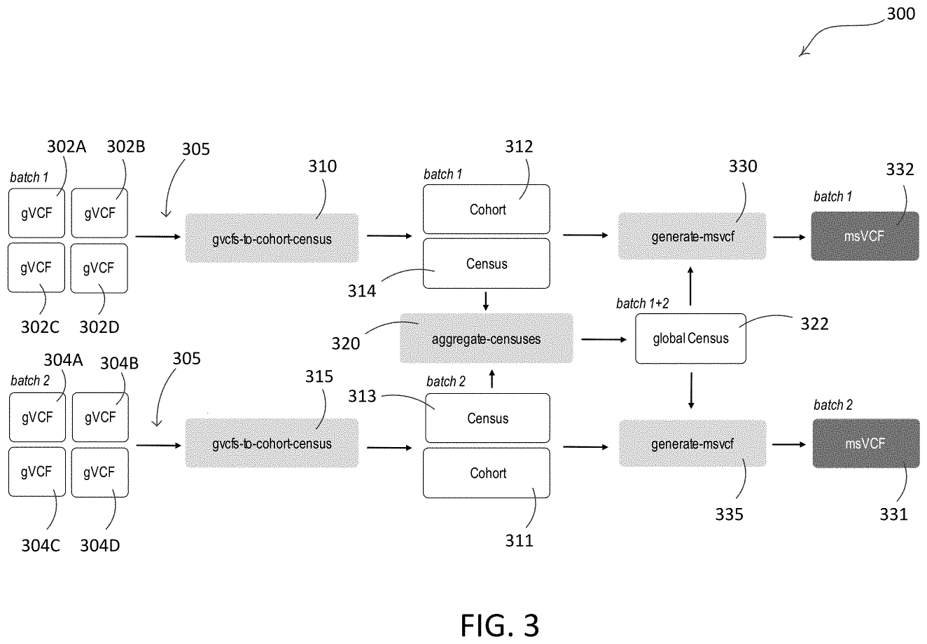

An iterative gVCF genotyper is described herein that may be implemented on one or more computing devices (e.g., one or more server devices 102 and/or client device 108 shown in FIG. 1) to iteratively aggregate variant data across a plurality (e.g., hundreds of thousands or more) of samples. FIG. 3 depicts an example process 300 that may be implemented by an iterative gVCF genotyper on one or more computing devices (e.g., such as the server device 102, the client device 108, and/or the sequencing device 114 shown in FIG. 1) to perform iterative gVCF genotyping, as described herein. The iterative gVCF genotyper may be implemented by a processor via computer-readable or machine-readable instructions stored in and accessed from memory. For example, the process 300, or portions thereof, may be implemented in computer-executable instructions that are stored in memory and executed by a processor at the one or more computing devices. The process 300, or portions thereof, may be performed to efficiently aggregate and/or store variant data. The process 300, or portions thereof, may be performed at a single computing device or may be distributed across multiple computing devices (e.g., multiple server(s), sequencing device(s) and/or client computing device(s)). The process 300, or portions thereof, may reduce the amount of processing resources and/or storage space (e.g., memory) used by the computing device(s) during a variant analysis. The process 300 may enable high throughput for sequencing analysis of gVCF files.

The iterative gVCF genotyper may receive per sample gVCF data as an input. For example, the iterative gVCF genotyper may receive a gVCF file for each sample. As described in FIG. 3, at 305, one or more batches of sample data may be received in batches of gVCF files, such as gVCF files 302A, 302B, 302C, 302D in batch 1 and/or gVCF files 304A, 304B, 304C, 304D in batch 2. Each of the one or more batches may have a batch size of about one thousand individuals (or samples). A sample size in a batch may be between one and a few thousand or another number. Each gVCF file 302A, 302B, 302C, 302D in batch 1 may include a summary of genome variant data that relates to a single sample. The gVCF files 302A, 302B, 302C, 302D may be received from a first sequencing device at a first site. Each gVCF file 304A, 304B, 304C, 304D in batch 2 may include a summary of genome variant data that relates to a single sample. The gVCF files 304A, 304B, 304C, 304D may be received from a second sequencing device at a second site. Genome variant data may be used interchangeably with genotype variant data.

The genome variant data may include a NON_REF field that indicates whether there are any possible alternative allele at a respective location. The genome variant data may include a LowQual field that indicates whether a record is low quality. The genome variant data may include an allelic depth (AD) field that indicates allelic depths for the ref and alt alleles in the order listed. The genome variant data may include a DP field that indicates an approximate read depth (e.g., reads with MQ=255 or with bad mates may be filtered). The genome variant data may include a GQ field that indicates a genotype quality associated with the record. The genome variant data may include a GT field that indicates a genotype associated with the record. The genome variant data may include a MIN DP value that indicates a minimum DP observed within the GVCF block. The genome variant data may include a PGT value that indicates physical phasing haplotype information that describes how the alternate alleles are phased in relation to one another. The genome variant data may include a PID value that indicates physical phasing ID information, where each unique ID within a given sample (e.g., but not across samples) connects records within a phasing group. The genome variant data may include a PL value that indicates normalized, Phred-scaled likelihoods for genotypes as defined in the VCF specification. The genome variant data may include an SB value that indicates per-sample component statistics which comprise the Fisher's Exact Test to detect strand bias.

The genome variant data may include a minimum amount of coverage observed at any one site within a block of records. The genome variant data may include a BaseQRankSum value that indicates a Z-score from Wilcoxon rank sum test of Alt Vs. Ref base qualities. The genome variant data may include a ClippingRankSum value that indicates a Z-score From Wilcoxon rank sum test of Alt vs. Ref number of hard clipped bases. The genome variant data may include a DP value that indicates an approximate read depth (e.g., some reads may have been filtered). The genome variant data may include a DS value that indicates which samples were downsampled. The genome variant data may include an END value that indicates a stop position of the interval. The genome variant data may include an ExcessHet value that indicates a Phred-scaled p-value for exact test of excess heterozygosity. The genome variant data may include an InbreedingCoeff value that indicates an inbreeding coefficient as estimated from the genotype likelihoods per-sample when compared against the Hardy-Weinberg expectation. The genome variant data may include a maximum likelihood expectation allele count (MLEAC) value that indicates a maximum likelihood expectation (MLE) for the allele counts (e.g., not necessarily the same as the AC), for each ALT allele, in the same order as listed. The genome variant data may include a maximum likelihood expectation allele frequency (MLEAF) that indicates a maximum likelihood expectation (MLE) for the allele frequency (e.g., not necessarily the same as the AF), for each ALT allele, in the same order as listed. The genome variant data may include an MQ value that indicates an RMS Mapping Quality. The genome variant data may include an MQRankSum value that indicates a Z-score From Wilcoxon rank sum test of Alt vs. Ref read mapping qualities. The genome variant data may include a RAW value that indicates raw data for RMS mapping quality. The genome variant data may include a ReadPosRankSum value that indicates a Z-score from Wilcoxon rank sum test of Alt vs. Ref read position bias.

The iterative gVCF genotyper may perform aggregation in an “end-to-end mode”, for example, when a single batch (e.g., only a single batch) of samples is available. The end-to-end mode may be implemented when there is one batch samples (e.g., the first batch). In the “step-by-step” mode, the gVCF files may be aggregated into cohort and census files, followed by generation of an msVCF file from cohort and census file of the same batch. Aggregation may not be performed in the “end-to-end” mode, where the gVCF genotyper may write a multi-sample VCF file without writing cohort and census files. The “end-to-end” process may not be iterative, as the cohort and census files may not be iteratively generated.

The iterative gVCF genotyper may perform aggregation in a “step-by-step mode,” for example, when multiple batches of samples are available, such that files are generated and information is aggregated for each batch. In the step-by-step mode, when multiple batches of samples are available, the iterative gVCF genotyper may execute a step for aggregation of gVCFs into cohort and census files for each batch. Once aggregation has been performed for each batch, the census files from the batches may be aggregated in the next step into a global census file. An msVCF may be generated using the global census file, the cohort file, and the census file for each batch, as described herein.

As shown in FIG. 3, the iterative gVCF genotyper may use the process 300 to aggregate a batch of gVCF files into a generated cohort file and a generated census file. At 310, the batch of gVCF files 302A, 302B, 302C, 302D may be converted into a cohort file 312 and a census file 314. At 315, the batch of gVCF files 304A, 304B, 304C, 304D may be converted into a cohort file 311 and a census file 313. The formats of the cohort files 311, 312 and the census files 313, 314, together, may enable users to extract variant data from the publicly available standard gVCF file format, and ingest and compress the data for large scale aggregation.

The cohort files 311, 312 and the census files 313, 314 may each be smaller files in size than the gVCF files from which they are generated. The cohort file 311 and the census file 313 may each comprise subsets of data from the respective batches of gVCF files 304A, 304B, 304C, 304D from which they are generated. The cohort file 312 and the census files 314 may each comprise subsets of data from the respective batches of gVCF files 302A, 302B, 302C, 302D from which they are generated. For example, the cohort files 311, 312 may each include at least the AD field, the GQ field, the FT field, the QL field, the PL field, and the RAGT field from the batches of gVCF files from which they are generated. The iterative gVCF genotyper may be configured to store single sample or multi-sample level variant data in the cohort files 311, 312. For example, the iterative gVCF genotyper may generate a cohort file 311, 312 for each batch of samples (e.g., batch 1, batch 2, etc.) by aggregating data from gVCF files associated with each of the samples in a respective batch for the subset of fields in the cohort files 311, 312.

The cohort files 311, 312 may be generated in a condensed data format that is used to store gVCF data in multiple samples. An example cohort file of a batch with 12 samples may be represented by the following,

```
{
  “DP”: {“32”: [0], “30”: [1, 7], “42”: [2], “38”: [3, 8], “41”: [4], “35”:
[5], “37”: [6], “21”: [9, 10, 11]},
  “GQ”: {“50”: [0, 6, 10, 11], “47”: [1, 8], “123”: [2], “110”: [3], “119”:
[4], “49”: [5, 9], “87”: [7]}
  “MQ”: {“250”: [0, 1, 2, 3, 4, 5, 6, 7, 8, 9, 10, 11]},
  “FT”: {“PASS”: [0, 1, 2, 3, 4, 5, 6, 7, 8, 9, 10, 11]},
  “QL”: {“323”: [0], “235”: [1], “173”: [2], “160”: [3], “169”: [4], “312”:
[5], “359”: [6], “137”: [7], “328”: [8], “255”: [9], “259”: [10], “237”: [11]},
  “PL”: {“323.1,300.26,53,273.1,4.3489e-05,53,450,305.84,307.33,450”: [0],
“234.7,184.7,48.807,448.53,7.8953e-05,53,450,218.93,448.53,450”: [1],
“173.36,123.36,0,450,158.13,450”: [2], “159.81,109.81,0,450,144.58,450”: [3],
“168.54,118.54,0,450,153.31,450”: [4], “311.97,261.97,51.169,388.78,5.4879e-
05,53,450,293.77,387.06,450”: [5], “358.65,345.07,52.999,308.65,4.3489e-
05,53,450,345.08,342.72,450”: [6], “136.99,86.991,0,450,121.76,450”: [7],
“327.73,277.73,47.841,450,9.319e-05,53,450,273.29,450,450”: [8],
“255.29,205.29,51.451,217.94,5.2808e-05,53,430.27,223.68,252.53,416.89”: [9],
“258.66,208.66,52.073,220.18,4.8666e-05,53,435.98,227.26,254.8,422.79”: [10],
“236.62,186.62,52.889,190.92,4.4006e-05,53,400.8,203.18,225.31,385.59”: [11]},
  “AD”: {“0,16,16,0”: [0], “0,20,10,0”: [1], “0,42,0”: [2], “0,38,0”: [3],
“0,41,0”: [4], “1,20,14,0”: [5], “0,18,19,0”: [6], “0,30,0”: [7], “0,23,15,0”:
[8], “0,12,9,0”: [9, 10], “0,11,10,0”: [11]},
  “RAGT”: {“TACAC:TAC,T,.:1/2”: [0, 6, 11], “TACACAC:T,TACAC,.:1/2”: [1],
“TACAC:T,.:1/1”: [2, 7], “TAC:T,.:1/1”: [3, 4], “TACACAC:T,TAC,.:1/2”: [5],
“TACACAC:TAC,T,.:1/2”: [8], “TACACAC:TACAC,T,.:1/2”: [9, 10]},
}
```

As shown in the above example, each field in the cohort file may include a summary of metrics or values corresponding to a similar field that is in each of the aggregated gVCF files (e.g., such as the gVCF files 302A, 302B, 302C, 302D for cohort 312 and 304A, 304B, 304C, 304D for cohort 311). For example, as illustrated in the “DP” field, the iterative gVCF genotyper may identify each of the unique metrics in the “DP” field for each gVCF file in the batch. Each of the unique metrics or values in the “DP” field in the example above may include the metric “32”, metric “30”, metric “42”, metric “38”, metric “41”, metric “35”, metric “37”, and metric “21”. Each metric may be followed by an array of integers that include the integer identifier of the gVCF files or samples that include the metric in the corresponding field in the gVCF file. For example, the gVCF files having the identifier “1” and “7” include the metric of “30” in the “DP” field, the gVCF files having the identifier “2” includes the metric of “42” in the “DP” field, and so on. The metric or value may be stored in a text format in the cohort file. The integer identifiers of the gVCF files or samples corresponding to each metric may in the cohort file be stored as an integer array to save on size of the cohort file and allow for additional compression of the file, as described herein. The cohort files 311, 312 may be used compute and store a count of metrics on number of variants and/or which positions in the genome are hom-ref or no data.

The gVCF file format includes redundant information. In each gVCF file of one sample, the same value of metrics in each field (e.g., FILTER, GT or ALT, NON_REF) may be repeated in string format in different records. When aggregated in whole, the gVCF files (e.g., such as the gVCF files 302A, 302B, 302C, 302D, 304A, 304B, 304C, 304D) may comprise multiple repetitive values for various metrics. For example, in the example provided above, the gVCF file having the identifier “1” and the gVCF file having the identifier “7” would each have to be stored with a text field comprising the field value “DP” and the metric of “30”. The above example for formatting the cohort file may allow for avoiding of redundancies. For example, the formatting of the cohort file may allow for storage of summary information from the batch of gVCF files for a predefined subset of the fields in the gVCF file an efficient way. As the number of samples being sequenced continues to grow, the formatting of the cohort file described herein may continue to provide savings in storage and processing resources when analyzing the sequencing data for genome variant data and other genotype data. The cohort files 311, 312 are also more stable than the gVCF files because the fields are fixed. The fixed fields in the gVCF files may be a subset of the fields that include data in the gVCF files that may be rarely changed by a user. The cohort files 311, 312 are less redundant than the storage of each of the gVCF files in the batch, because they include less repetition of the field identifiers and values across the samples. Instead, one particular region of each of the cohort files 311, 312 may be represented the data across each of samples in the batch of gVCF files for efficient storage. In the cohort files 311, 312, the data may be ordered by unique metrics values, and for each unique value, the sample identifier value may be stored to avoid repeating the same value across multiple records in multiple samples.

The iterative gVCF genotyper may further analyze the data in each of the cohort files 311, 312 to generate a corresponding census file 313, 314. The census files may have a smaller file size than the cohort files 311, 312 from which the census files 313, 314 are generated. The census files 313, 314 may be used to store summary statistics of the variants (e.g., each of the variants) and/or reference blocks among samples in the cohort (e.g., batch) data represented in the corresponding cohort files 311, 312. An example census file of the batch with 12 samples may be represented by the following,

```
{
  “DP”: {“32”: 1, “30”: 2, “42”: 1, “38”: 2, “41”: 1, “35”: 1, “37”: 1, “21”:
3},
  “GQ”: {“50”: 4., “47”: 2, “123”: 1, “110”: 1, “119”: 1, “49”: 2, “87”: 1},
  “MQ”: {“250”: 12},
  “FT”: {“PASS”: 12},
  “QL”: {“323”: 1, “235”: 1, “173”: 1, “160”: 1, “169”: 1, “312”: 1, “359”: 1,
“137”: 1, “328”: 1, “255”: 1, “259”: 1, “237”: 1},
  “PL”: {“323.1,300.26,53,273.1,4.3489e-05,53,450,305.84,307.33,450”: 1,
“234.7,184.7,48.807,448.53,7.8953e-05,53,450,218.93,448.53,450”: 1,
“173.36,123.36,0,450,158.13,450”: 1, “159.81,109.81,0,450,144.58,450”: 1,
“168.54,118.54,0,450,153.31,450”: 1, “311.97,261.97,51.169,388.78,5.4879e-
05,53,450,293.77,387.06,450”: 1, “358.65,345.07,52.999,308.65,4.3489e-
05,53,450,345.08,342.72,450”: 1, “136.99,86.991,0,450,121.76,450”: 1,
“327.73,277.73,47.841,450,9.319e-05,53,450,273.29,450,450”: 1,
“255.29,205.29,51.451,217.94,5.2808e-05,53,430.27,223.68,252.53,416.89”: 1,
“258.66,208.66,52.073,220.18,4.8666e-05,53,435.98,227.26,254.8,422.79”: 1,
“236.62,186.62,52.889,190.92,4.4006e-05,53,400.8,203.18,225.31,385.59”: 1},
  “AD”: {“0,16,16,0”: 1, “0,20,10,0”: 1, “0,42,0”: 1, “0,38,0”: 1, “0,41,0”:
1, “1,20,14,0”: 1, “0,18,19,0”: 1, “0,30,0”: 1, “0,23,15,0”: 1, “0,12,9,0”: 2,
“0,11,10,0”: 1},
  “RAGT”: {“TACAC:TAC,T,.:1/2”: 3, “TACACAC:T,TACAC,.:1/2”: 1,
“TACAC:T,.:1/1”: 2, “TAC:T,.:1/1”: 2, “TACACAC:T,TAC,.:1/2”: 1,
“TACACAC:TAC,T,.:1/2”: 1, “TACACAC:TACAC,T,.:1/2”: 2}
}
```

When a large number of samples are available, a user may divide samples into multiple batches each with similar sample size (e.g., 1000 samples). As shown in the example above, the census files 313, 314 may include a sample count for each unique metric or value in each field in the census file. The sample count may be calculated and stored in the census file instead of a sample identifier for each metric, as illustrated in the example cohort file. As shown in the above example, each field in the census file may include a summary of metrics corresponding to a similar field that is in the corresponding cohort file. For example, as illustrated in the “DP” field, the iterative gVCF genotyper may calculate the total number of unique gVCF identifiers for each metric in the “DP” field of the cohort file. Each of the unique metrics in the “DP” field in the example census file above may include the metric “32”, metric “30”, metric “42”, metric “38”, metric “41”, metric “35”, metric “37”, and metric “21”. Each metric may be followed by an integer value of the total number of unique gVCF identifiers having the metric in the corresponding field. For example, the total number of the gVCF files in the batch having the metric of “32” is “1”, the total number of the gVCF files in the batch having the metric of “30” is “2”, and so on. The metric may be stored in a text format. The summary of identifiers of the gVCF files corresponding to each metric may be stored as an integer value to save on size of the census file and allow for additional compression of the file, as described herein. The storage and processing of the sample counts in the census files 313, 314 may be smaller than the list of sample identifiers listed in the metrics in the cohort files 311, 312, which may remove the sample level information (or identifiers) from the census data in the census files that may be used to aggregate genome variant data and other genotyping data at a higher level. Thus, the census files 313, 314 may require less memory storage and processing resources to analyze than the cohort files 311, 312.

The cohort file and the census file may be file formats that can be efficiently stored, analyzed, and/or communicated. In an example, the cohort file and the census file may be file formats that can be efficiently compressed, indexed, and/or queried by genomic regions using genomic algorithms and/or compression algorithms. The cohort file may store, per each genomic region (position with variant or blocks without variant) multiple fields extracted from gVCF files. For each field, the cohort file may store multiple values (e.g., metrics) and for each value (e.g., metrics), the identifier of each sample with a particular value of that field, as further described herein. The cohort file stores data per field across multiple samples, rather than per sample with multiple fields, therefore the query of one field across large number of samples is more efficient. The census file may store fields in similar structure, but instead of storing identifier of samples for a particular value of one field, the census file may store the number of samples, which allows for continuous accumulation of sample count for different fields as batches of samples are aggregated. Storing sample counts instead of sample identifiers also reduces census file size allowing for aggregation of a plurality (e.g., thousands) of batches of samples. In both cohort and census files, the data may be further be encoded using bit encoding or hash encoding to reduce the data footprint, the encoded binary data may be further compressed using gzip or other public compression algorithm into compressed binary blocks before being stored on disk. The compressed blocks may be indexed to allow for random access of genomic regions. Being able to efficiently compress, index, and query files by genomic regions may be features that may be made accessible when aggregating variant data at scale. The use of these compressed files may reduce the hardware disk footprint, which may reduce the local workflow and/or workflow in the Cloud since data transfer can incur significant ingress and egress costs as well as storage costs. The use of these compressed files may allow for more efficient transfers of data and lower costs for remote storage given the data structure of the files and data structures described herein.

When the census files 313, 314 are generated for a plurality of batches, the iterative gVCF genotyper may aggregate at 320 the census files 313, 314 into a global census file 322. The census files 313, 314 may be aggregated from multiple batches (e.g., batch 1 and batch 2) from sequencing devices at different sites to generate the global census file 322. It will be appreciated that although the example shown in FIG. 3 has two batches, the process 300 is not limited to two batches. Instead, the process 300 may receive any number of batches of gVCF files from sequencing devices at any number of sites. The global census file 322 may include summary statistics for variant sites and non-variant genomic blocks for a single sample and/or across multiple samples (e.g., multiple samples on a single sequencer). Each variant record in the global census file 322 may include the RAGT statistics and the variant statistics from the global census file, the RAGT statistics and the variant statistics from the batch census files 313, 314 (e.g., for samples included in the per batch msVCF file), and other metrics (e.g., non-RAGT metrics) from other fields, such as FT, GQ, AD, PL (with sample identifiers) from the batch cohort files 311, 312. In one example, the global census file 322 may include an RAGT field that includes the RAGT statistics for different alleles that have been identified from the different sample data received from the batches of samples at different sites. The RAGT field may be a summary field used to determine each of the variant statistics (e.g., POS, REF, ALT, INFO fields) in summary fields in other filed (e.g., in the msVCF file described herein). From RAGT statistics, site statistics may be generated that are stored in the census files (e.g., per batch census files 313, 314 and global census file 322). The site statistics may include the number of samples with genotype, without genotype (but with coverage) and without coverage, the total number of samples, the alternative allele count, and allele order, and/or mapping between global alternate alleles and per sample ALT alleles as stored in the RAGT.

The processing of the data received in the gVCF files (e.g., the gVCF files 302A, 302B, 302C, 302D, 304A, 304B, 304C, 304D) may be executed in parallel across multiple samples to generate the global census file 322 across multiple samples. For example, the cohort file 312 and the census file 314 may be generated from batch 1 of the gVCF files 302A, 302B, 302C, 302D, while the cohort file 311 and the census file 313 may be generated from batch 2 of the gVCF files 304A, 304B, 304C, 304D using parallel processing as described herein. The global census file 322 may include one or more secondary metrics, for example, such as allele counts, percentage of samples without sequencing coverage, percentage of samples without reliable genotypes, and/or the like. Generating the global census file 322 may scale to aggregate a large number (e.g., thousands) of batches, in a more efficient way, than aggregating gVCF files from the (e.g., all of the) batches. For example, instead of having to open each gVCF file and search the files to find a piece of information (e.g., summary statistic or secondary metric), the global census file 322 may aggregate the summary statistics and secondary metrics in one place.

When the global census file 322 has been generated, the iterative gVCF genotyper may generate at 330, 335 a respective multi-sample VCF (msVCF) file 331, 332 for each batch. The msVCF files 331, 332 may be generated using the global census file, a respective one of the cohort files 311, 312, and/or a respective one of the census files 313, 314. For example, the msVCF file 332 may be generated (e.g., for batch 1) using the cohort file 312, the census file 314, and the global census file 322. The msVCF file 332 may be generated (e.g., for batch 2) using the cohort file 311, the census file 313, and the global census file 322. The process 300 may end after generation of the msVCF files 331, 332. The msVCF files 331, 332 may include batch-specific genome variant data and/or other genotype data, as well as global census data that includes genome variant data and/or other genotype data identified from samples taken at other sites. Examples are the global set of variant sites and the variant alleles. genotypes called for each sample, read depth for variant alleles and hom-ref positions. Likelihoods and quality scores for alleles

In an example, the msVCF file may include one or more global statistics, for example, allele frequencies, a read depth for variant alleles and/or home-ref positions, a number of samples with or without genotypes, and a number of samples without coverage. The msVCF file may include a likelihood and/or quality score for alleles. Similar statistics among samples in the batch are also included. An msVCF file may be generated for each batch of samples, to facilitate data read/write efficiency, data transfer, storage, and/or querying. The number of records in each msVCF file may be the same across all batches, which allows for quick look up of the same variants across multiple batches.

The fields in each of the msVCF files 331, 332 may include a summary of genotypes and/or samples. For example, the fields in the msVCF files 331, 332 may include an allele count in genotypes, a total number of alleles in called genotypes, a total number of samples, a total number of samples with called genotypes, a total number of samples with unknown genotypes, and/or a total number of samples with no coverage. The metrics that are calculated for each of these fields may be generated in multiple versions. For example, the metrics for each of these fields may be calculated once per batch to provide a local version of the metric and once for each of the samples in the cohort file to provide a global version of the metric.

As will be understood, the DNA sequencing process may have a random component. A sequencing run may not yield any information (=coverage from sequencing reads) for a small set of positions of the genome. This may be captured by a “no coverage” metric. The number of these positions may be used to distinguish from other positions. For example, the variant information or statistics may include variant data indicating that a position has a DNA mutation, home-ref data indicating that a position has no DNA mutation, or no-coverage data that indicates that the variant information of the position is unknown. The gVCF variant files may include this information for a specific sample. The gVCF file may include, for each position in the genome, the variant information indicating that the position has a DNA mutation, home-ref data indicating that the position has no DNA mutation, or no-coverage data that indicates that the variant information of the position is unknown. The census files 313, 314 may store this information for a batch of samples and by merging per-batch census files into the global census file 322 and then sending the global census back to each batch during msVCF writing to include these metrics in each per-batch msVCF file.

The msVCF file may be a standardized public data format which enables users to run down-stream analysis, for example, genome-wide association studies (or GWAS), imputation and phasing, gene burden analysis, rare variant discovery, population specific allele frequencies, population substructure analysis, and estimating pathogenicity of mutation and classifying deleterious/benign variants in clinical analysis. The msVCF file may enable access to and querying of aggregated variant data (e.g., in a batch). The msVCF file may include variant site level information both within the current batch and global across all batches of samples from sequencing devices at each site. The variant site level information may include, allele count, allele frequency, the total number of samples, the number of samples without coverage, the number of samples with coverage but not reliable genotypes and the number of samples with genotype. A user may prefer to use the msVCF file to access and query the aggregated variant data, for example, instead of other file formats. An example msVCF file of a batch may be represented by the following,

chr21 9053774. TACAC
AACAC,CACAC,T,TAC,TACACAC,TACACACACACAC,<NON_REF> . PASS
AC=0,338,1,1,173,2,0;AN=5218;NS=3202;NS_GT=2609;NS_NOGT=297;NS_NODATA
=296;
GAC=0,338,1,1,173,2,0;GAN=5218;GNS=3202;GNS_GT=2609;GNS_NOGT=297;GNS
_NODATA=296
GT:GQ:AD:FT:LPL:LGT:LAA
0/0:14:18,0,0,0,0,0,0,0:PASS:0,14,59:R;X;0/0:7
. . . . . .
.............
0/0:2:0,0,2,0,0,0,0,0:DRAGENHardQUAL:37,6,0,37,6,37:T;A,X;0/0:2,7
./.:0:0,0,0,0,0,0,0,0:LowDepth;LowGQ:0,0,0:R;X;./.:7
2|2:4:0,0,2,0,0,0,0,0:DRAGENHardQUAL:46,6,0,70,6,70:T;C,X;1|1:2,7
0/5:12:15,0,0,0,0,14,0,0:PASS:27,0,14,326,339,665:T;TAC,X;0/1:5,7
0/3:11:21,0,0,14,0,0,0,0:PASS:24,0,14,383,603,986:TACAC;T,X;0/1:3,7
...

When a global census file (e.g., such as the global census file 322) has already been generated and a batch of samples becomes available from the sequencer, the iterative gVCF genotyper may aggregate the sample data in the batch into a cohort file (e.g., such as the cohort files 311, 312) and a census file (e.g., such as the census files 313, 314) for the batch. The iterative gVCF genotyper may then aggregate the census file from the batch with the global census file from the (e.g., all) previous batches, for example, to generate an updated global census file.

After the global census file is updated (e.g., with new variant sites discovered and/or variant statistics updated at existing variant sites), the iterative gVCF genotyper may again generate an msVCF file for each batch of samples using the cohort file for the batch, the census file for the batch, and the updated global census file. The msVCF file may include the variants and alleles discovered in each of the samples from the each of the batches received from genotyping devices across sites. The msVCF files 331, 332 may then include batch-specific genome variant data and/or other genotype data, as well as updated global census data that includes genome variant data and/or other genotype data identified from samples taken at other sites. The iterative gVCF genotyper may provide an iterative population-based analysis option to jointly analyze samples from unrelated individuals, for example, samples become available for analysis.

The gVCF files may follow block compression and may have certain columns that are used to indicate which chromosomes the data belongs to and one or more columns that indicate start/end points. The process 300 may incorporate a hybrid of binary and ASCI or string compression and/or serialization to minimize the amount of storage space and/or processing resources required to store the variant data and generate msVCF files.

The iterative gVCF genotyper may use the example process 300 to aggregate sample data from multiple sequencing devices at multiple sites into an existing data file, such as the msVCF file. The iterative gVCF genotyper may use the example process 300 to incrementally aggregate newly available genome variant data and other genotype data from batches of sample data with genome variant data and other genotype data available in prior batches, for example, without having to redo the analysis for the previously available (e.g., and aggregated) batches. For example, the iterative gVCF genotyper may incrementally aggregate the genome variant data and/or other genotype data as it becomes available. The iterative gVCF genotyper may be scalable and may be executed on a plurality of computing platforms, for example, such as a cloud platform, a high performance cluster, and/or a single server.

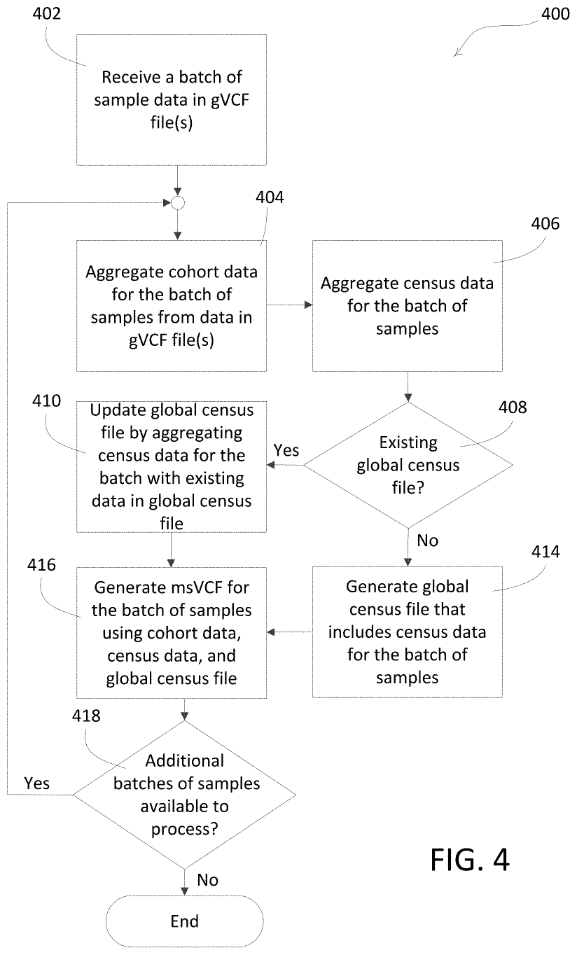

FIG. 4 is a flowchart of an example method 400 for processing batches of sample data from a sequencing device for generating one or more msVCF files. The example method 400 may be performed by one or more computing devices. For example, the example method 400 may be executed by one or more processors executing computer-readable or machine-readable instructions stored in memory at the one or more computing devices for operating an iterative gVCF genotyper. The example method 400 may be executed in response to input received from a user in software for building one or more msVCF files. One or more portions of the example method 400 may be executed locally by the sequencing application 110 executing on the client device 108 and/or remotely by the sequencing system 104 executing on the shown in FIG. 1. One or more portions of the example method 400 may be executed remotely on a cloud-based environment or other remote computing device or devices, such as by the sequencing system 104 on the server device(s) 102 shown in FIG. 1. Though the example method 400 may be described herein as being performed by a single device, the example method 400 may be distributed across multiple devices. Similarly, though the example method 400 may be described herein as being performed by a single processing entity, the example method 400 may be performed using parallel processing of batches of sample data, as described herein. For example, one or more portions of the example method 400 may be implemented by a virtual machine or compute node having separate resources for performing the one or more portions of the example method 400.

As illustrated in FIG. 4, the method 400 may begin at 402 when a batch of sample data is received in one or more gVCF files. The batch of sample data may be received from a sequencing device capable of processing the samples to generate the sample data or another device capable of generating and/or transmitting gVCF files. The sample data may include genome variant data and/or other genotype data. For example, the sample data may include data in the one or more fields of the gVCF file as described herein. At 404, the processor of the computing device may aggregate cohort data for the batch of samples from the sample data in the gVCF files. The cohort data may include the sample data in a subset of fields in the gVCF files. The processor of the computing device may aggregate census data for the batch of sample data at 406. The census data may include variant summary statistics and reference blocks of the sample data in the batch. The cohort data and the census data may be stored in separate file types, databases, or other data structures.

The processor of the computing device may determine, at 408, whether there is an existing global census file for aggregating global census data corresponding to the sample data from each of the batches being received from sequencing devices at multiple sites. If a global census file fails to exist or fails to exist for aggregating the sample data in the received batch, the processor of the computing device may generate a global census file, at 414, that includes census data for the batch of samples. The global census file may include the variant summary statistics that are global metrics (e.g., allele counts, allele frequencies, etc,) for the sample data from each of the batches of samples being received. If the global census file already exists, the processor of the computing device may update the global census file, at 410, by aggregating census data for the batch with the already existing data in the global census file. This may include adding census data for the batch that is received in the sample data and/or generating variant summary statistics using the sample data in the batch and adding the variant summary statistics to the global census file.

At 416, the processor of the computing device may generate an msVCF file for the batch being processed. The msVCF file may be generated for the batch using the cohort data, the census data for the batch, and/or the global census file. Each variant record in the global census file may include the RAGT statistics and the variant statistics from the global census file, the RAGT statistics and the variant statistics from the batch census files (e.g., for samples included in the per batch msVCF file), and other metrics (e.g., non-RAGT metrics) from other fields, such as FT, GQ, AD, PL (with sample identifiers) from the batch cohort files. The RAGT statistics and the variant statistics may include variant positions, variant alleles, sample genotypes and/or qualities of variant calls, and/or other RAGT statistics and the variant statistics. The computing device may store (e.g., locally, remotely, and/or in the cloud) the msVCF file. After the msVCF file(s) are generated at 416, the process 400 may determine whether there are additional batches of sample data available from the sequencer or other computing device, at 418. If there are additional batches to be processed, the procedure may return to 404 to aggregate the cohort data and census data for the batch and proceed as described herein. If there are no additional batches of sample data available from the sequencer, the method 400 may end. The cohort file, census file, global census file, gVCF file, and/or msVCF file may be stored in memory in a manner to improve or optimize memory usage. For example, data may be stored in memory using hash codes and/or hash maps, as further described herein.

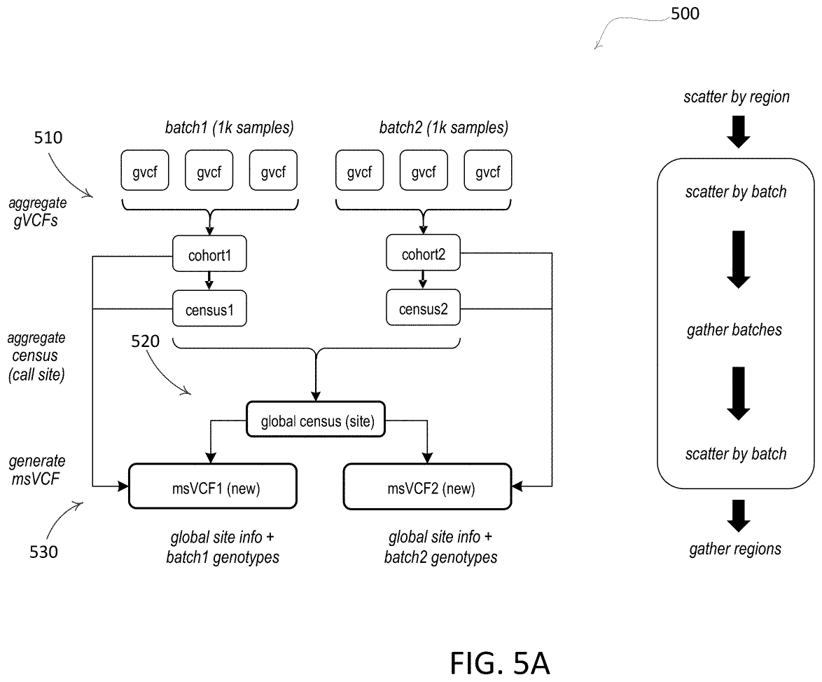

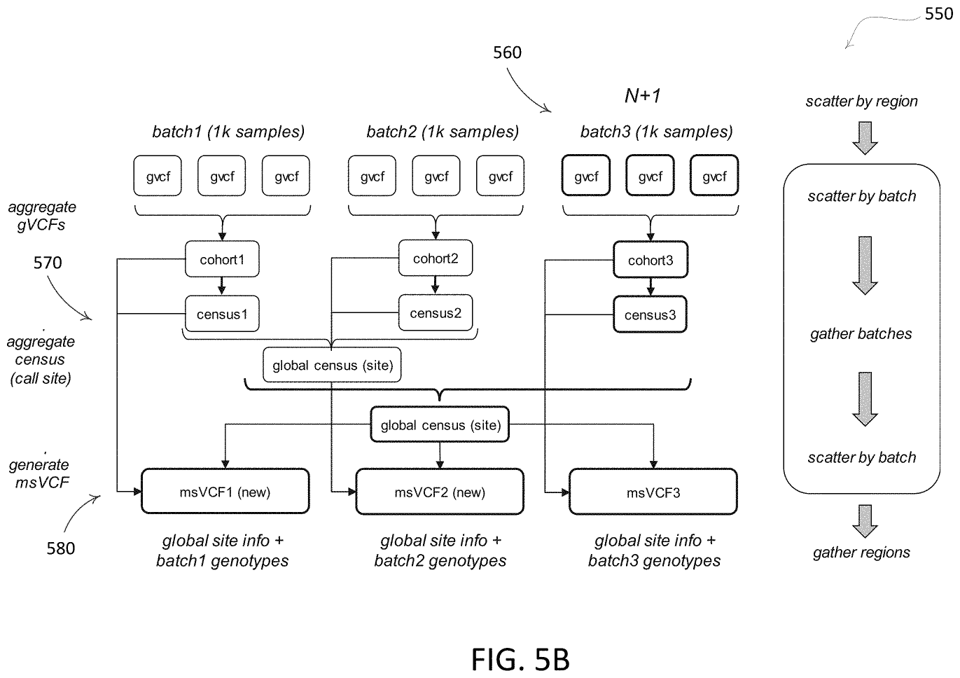

As illustrated in the method 400 shown in FIG. 4 and otherwise described herein, a global census file may be generated from multiple batches of sample data received from sequencing devices at different sites and may be updated as additional batches of sample data are received from sequencing devices at additional sites. FIGS. 5A and 5B illustrate example processes 500, 550 illustrating how the global census file may be generated and updated, and how msVCF files may be generated and/or updated in response to the sample data in the updated global census file. FIG. 5A illustrates an example scenario when there are two batches of gVCF files available at the same time for processing. FIG. 5B illustrates a further example when an additional batch of gVCF files are later available. The example processes 500 shown in FIG. 5A may be performed by an iterative gVCF genotyper to initially generate a global census file. The example processes 550 shown in FIG. 5B may be performed by an iterative gVCF genotyper for updating the global census file as additional batches of sample data are received. The processes 500, 550 may be performed by one or more computing devices. For example, processes 500, 550 may be executed on multiple computing devices where each of the computing devices executes one independent region or chromosome in the genome. The processes 500, 550 may be executed by one or more processors executing computer-readable or machine-readable instructions stored in memory at the one or more computing devices. The processes 500, 550 may be executed in response to input received from a user in software for building one or more msVCF files. One or more portions of the processes 500, 550 may be executed locally by the sequencing application 110 executing on the client device 108 shown in FIG. 1. One or more portions of the processes 500, 550 may be executed remotely on a cloud-based environment or other remote computing device or devices, such as by the sequencing system 104 on the server device(s) 102 shown in FIG. 1. Though the processes 500, 550 may be described herein as being performed by a single device, the processes 500, 550 may be distributed across multiple devices. Similarly, though the processes 500, 550 may be described herein as being performed by a single processing entity, the processes 500, 550 may be performed using parallel processing of batches of sample data, as described herein. For example, one or more portions of the processes 500, 550 may be implemented by a virtual machine or compute node having separate resources for performing the one or more portions of the processes 500, 550.

As shown in FIG. 5A, the processor of the computing device executing the iterative gVCF genotyper may receive at 510 two or more batches of sample data. For example, the two or more batches of sample data may be available from the sequencing device capable of processing the samples to generate the sample data or another device capable of generating and/or transmitting gVCF files. Each batch of sample data may comprise data from a plurality of samples (e.g., 1000 samples or more). For example, the processor may receive at 510 gVCF files associated with a first batch of sequencing data and gVCF files associated with a second batch of sequencing data. The gVCF files associated with the first batch of sequencing data and/or the second batch of sequencing data may be split into shards of equal size, as described herein. Each of the shards may be processed using one of a plurality of computation nodes.

The processor executing the iterative gVCF genotyper may aggregate at 510 the sample data in each batch (e.g., in parallel) to generate a cohort file and a census file for each of the batches. For example, a first cohort file for a first batch (e.g., batch 1) may comprise a subset of fields in each of the gVCF files associated with the first batch (e.g., batch 1) of sequencing data. The census file for the first batch of sequencing data may comprise variant summary statistics (e.g., RAGT statistics) and/or hom-ref blocks of the first batch of sequencing data. A second cohort file for a second batch (e.g., batch 2) of sequencing data may comprise a subset of fields in each of the gVCF files associated with the second batch of sequencing data. The census file for the second batch of sequencing data may comprise variant summary statistics (e.g., RAGT statistics) and/or hom-ref blocks of the second batch of sequencing data. It should be appreciated that each of the cohort files generated for each respective batch may comprise the same subset of fields.

The processor executing the iterative gVCF genotyper may aggregate the census files at 520 from each of the batches to generate a global census file. The global census file may comprise census data from the batches of samples received from sequencing devices at different sites. The processor executing the iterative gVCF genotyper may generate at 530 msVCF files for the first and second batches of sequencing data. For each of the two or more batches, the iterative gVCF genotyper may use the cohort file for a respective batch, the census file for the respective batch, and the global census file to generate (e.g., in parallel) a msVCF file for the respective batch. One or more of the msVCF files may be used to perform a genome-wide sequencing analysis. Although the process 500 shows the initial global census file generated at 520 using variant summary statistics from two batches (e.g., batch 1 and batch 2), it will be appreciated that the process 500 is not limited to using two batches of sample data to generate the initial global census file. Instead, the process 500 may use many batches of sample data that are available (e.g., three or more batches). Each of the batches of sample data may be processed in parallel and/or used to generate the initial global census file at 520. It should also be appreciated that the census file may be updated by aggregating census files of subsequent batches of sample data with the previously generated global census file.

Turning now to the process 550 shown in FIG. 5B, the global census file may be updated in response to additional sample data received in additional batches of sample data. For example, the process 550 may begin after the process 500 has ended. The process 550 may receive an additional batch (e.g., batch 3) of sample data, for example, after the first two msVCF files have been generated.

The processor executing the iterative gVCF genotyper may aggregate at 560 the sample data in the additional batch (e.g., batch 3) to generate a cohort file and a census file for the additional batch. The iterative gVCF genotyper may aggregate at 570 the data across the batches of samples in the global census file, such that the global census file is updated based on the sample data in the additional batch of samples to generate an updated global census file. For example, the processor executing the iterative gVCF genotyper may generate an updated global census file by incorporating the data in the census file from the additional batch into the global census generated in the process 500. Being able to update the global census file and not having to aggregate again the census data from the previous census files may save processing resources (e.g., to generate a global census file with census data from all received batches and/or generate msVCF files for each batch).

The processor executing the iterative gVCF genotyper may use the cohort file for the additional batch, the census file for the additional batch, and the updated global census file to generate at 580 an msVCF file for the additional batch. The iterative gVCF genotyper may also use the cohort file for the existing batch, the census file for the existing batch and the updated global census file to generate at 580 updated msVCF files for each of the previously received batches. For example, the processor executing iterative gVCF genotyper may generate new msVCF files for each batch upon generation of the updated global census file (e.g., and receipt of additional batches of sequencing data).

In FIGS. 5A and 5B, the batches of gVCF files may be processed by two levels of parallelization by genomic region and sample batches. These two levels may be independent of each other. For example, the analysis of each genomic region can be done in parallel. For each genomic region, different batches can be further parallelized to generate the cohort files and census files. Once the cohort files and census files have been generated for each batch, the processor executing the iterative gVCF genotyper may aggregate the census files from each of the batches into a global census file. The generation of the global census file may be parallelized by region. Although FIGS. 5A and 5B indicate that each batch includes 1000 samples, it will be appreciated that each batch may include more or less than 1000 samples. Although FIG. 5B depicts three total batches, it will be appreciated that the iterative gVCF genotyper may receive gVCF files for any number of batches.

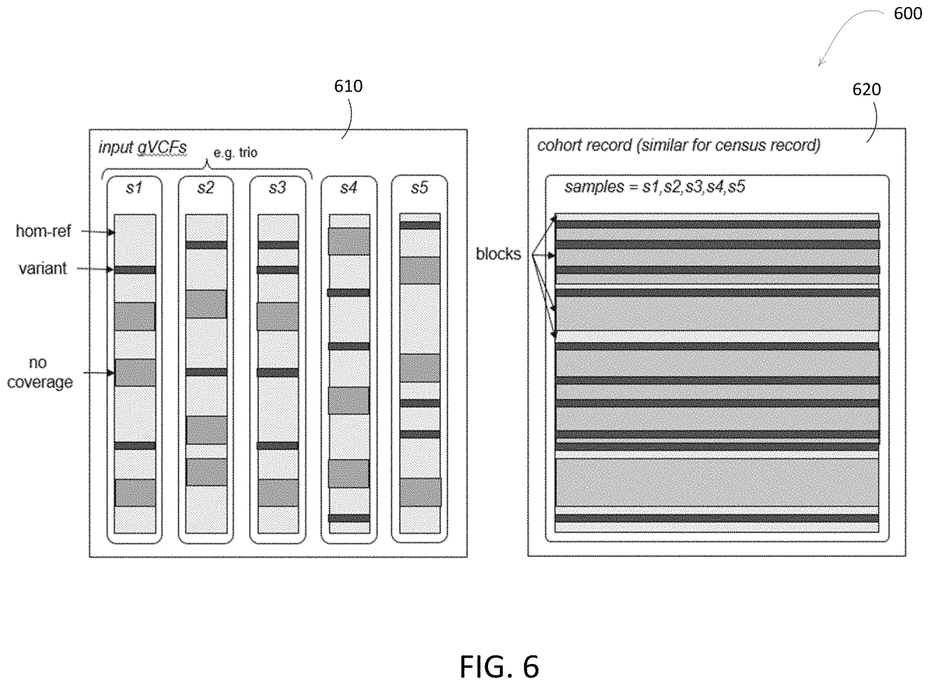

FIG. 6 depicts a graphical representation 600 of a cohort record generated for a batch to illustrate how similar types of data in multiple gVCF files may be aggregated in the cohort file. Similar types of information may also, or alternatively, be aggregated in the census file. As shown in FIG. 6, a set of input gVCF files 610 may be received by the processor executing the iterative gVCF genotyper. The batch in the example shown in the graphical representation 600 includes 5 samples. As shown in FIG. 6, the gVCF files 610 may include related samples, such as a trio of three samples which are related (e.g., father, mother, and child). Each sample may be associated with a respective gVCF file. Each of the gVCF files 610 comprises hom-ref data, variant data, and no coverage data disbursed in sections throughout the gVCF files. For example, each of the gVCF files 610 indicates hom-ref data, one or more variants, and no coverage areas for its respective sample. The hom-ref data, variant data, and no coverage data from the samples in the batch (e.g., set of input gVCF files) may be aggregated into a cohort file 620.

The cohort file 620 may be a single file that indicates the hom-ref, variant, and no coverage data for the batch (e.g., set of input gVCF files). In the cohort file 620, similar types of data (e.g., the hom-ref, variant, and no coverage data) may be aggregated in rows of the cohort file. The cohort file 620 may include blocks that include regions where each of the samples in the cohort have the same kind of records in the original gVCF (e.g., all hom-ref blocks or all no coverage). Although not shown, a census record may be generated for the batch and the census record may be similarly aggregated. Although FIG. 6 depicts the input gVCF files 610 for the batch comprising 5 samples, it will be appreciated that the more than 5 input gVCF files 610 may be received. For example, the number of input gVCF files 610 received may equal the number of samples in a batch.

The records in the cohort file may be grouped into regions with the same kind of record (e.g., hom-ref, no coverage, or variant) to reduce the data size and/or reduce processing resources for compression. In contrast, each per region cohort file may be broken into one record per genomic position, which would have adjacent records with the same values across samples.

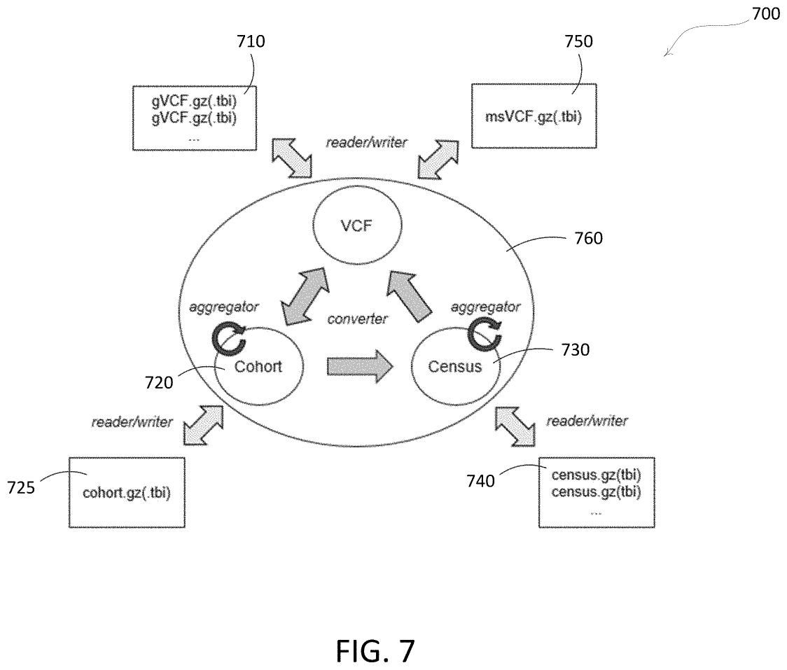

FIG. 7 depicts an example iterative process 700 that may be implemented by one or more computing devices (e.g., such as the server device 102, the client device 108, and/or the sequencing device 114 shown in FIG. 1) to generate multi-sample variant call files upon receipt of gVCF files associated with a batch. The one or more computing devices that performs the example iterative process 700 may be executing an iterative gVCF genotyper 760, as described herein. The iterative gVCF genotyper 760 may use the iterative process 700 to aggregate newly available batch/sample data into an existing cohort. The iterative gVCF genotyper 760 may use the iterative process 700 to incrementally aggregate newly available batches of sample data with previously available batches, for example, without having to redo the analysis for the previously available (e.g., and aggregated) batches. The iterative process 700, or portions thereof, may be performed to more efficiently store variant data in a census file, a cohort file, and/or a msVCF file. For example, the iterative process 700, or portions thereof, may be performed to aggregate gVCF files into cohort files and aggregating census files into a global census file.

The iterative process 700, or portions thereof, may be performed at a single computing device or may be distributed across multiple computing devices (e.g., multiple server(s), sequencing device(s) and/or client computing device(s)). The iterative process 700, or portions thereof, may reduce the amount of processing resources and/or storage space (e.g., memory) used by the computing device(s) during a variant analysis. The iterative process 700 may be used to process multiple batches of the sequencing run. The iterative process 700 may store the cohort files 720, the census files 730, the global census file 740, and/or the msVCF file(s) 750 locally or remotely.

The iterative process 700 may begin upon receipt of gVCF files 710 associated with a batch of sample data by the iterative gVCF genotyper 760. The iterative gVCF genotyper 760 may read the gVCF files 710 and identify a subset of the VCF fields in the gVCF files 710 for generating the cohort file 725. The iterative gVCF genotyper 760 may aggregate the gVCF data read from each of the gVCF files in a batch of gVCF files 710 in a batch. The gVCF file may comprise sample data from a sample of a sequencing run. The cohort data 710 may include a subset of the sample data in the gVCF files 710 that is identified in predefined fields. The cohort data 720 may be aggregated from each of the predefined fields in the batch of sample data read from the gVCF files 710 and the iterative gVCF genotyper 760 may write the subset of data to the cohort file 725. As described herein, the cohort data may include a summary of the unique identifiers for a gVCF file comprising a metric or value in each of the predefined fields in the batch of gVCF files 710.

The iterative gVCF genotyper 760 may convert the cohort data 720 to the census data 730 and write the census data to a census file 730 for the batch of sample data. The census data 730 may include a count of the number of unique gVCF files comprising a common metric or value for each of the predefined fields in the cohort file 725. The iterative gVCF genotyper 760 may aggregate the census data 730 from multiple census files 740 to generate a global census file. For each of the multiple batches, the iterative gVCF genotyper 760 may use the cohort data 720 and/or the census data 730 for a respective batch of samples and the data in the global census file to generate and write to an msVCF file 750 for the respective batch of samples, as described herein.

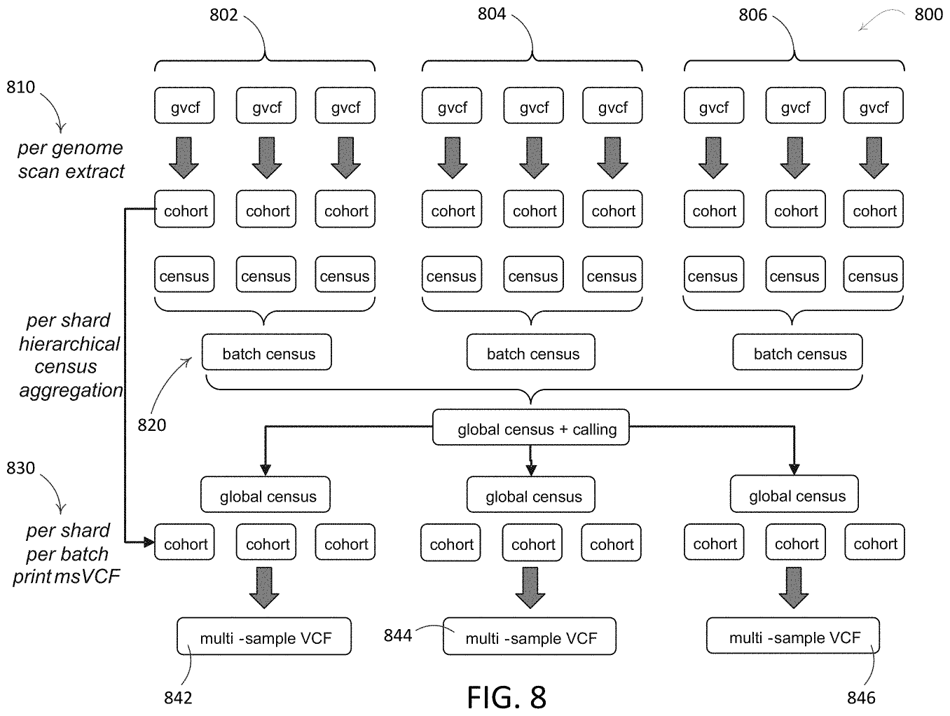

FIG. 8 depicts an example process 800 that may be implemented by one or more computing devices (e.g., such as multiple server devices 102, multiple client devices 108, and/or one or more server devices and one or more client devices 108 shown in FIG. 1) that may utilize parallel processing in the variant analysis of the sample data received in the gVCF files. For example, the example process 800 may incorporate parallel processing for processing each of the batches 802, 804, 806 of sample data from sequencing devices at different sites for generating one or more msVCF files. The process 800, or portions thereof, may be performed by an iterative gVCF genotyper distributed across multiple compute nodes to more efficiently store and process variant data in msVCF files. The process 800, or portions thereof, may reduce the amount of processing resources and/or storage space (e.g., memory) used by the computing device(s) during a variant analysis.

The iterative gVCF genotyper may utilize at least one processor and/or at least one memory to perform parallel processing using multiple compute nodes or virtual machines. The iterative gVCF genotyper may assign processing resources, as described herein, to enable to parallel processing being described. The iterative gVCF genotyper may split each sample (e.g., each gVCF file) in a batch (e.g., such as batch 802, batch 804, and/or batch 806) into a separate shard. When each sample is assigned to a shard, samples may be freely grouped in a defined group (e.g., such as a subpopulation or case/control group(s)). Each shard may be of equal or different size. Each shard may be processed by a separate compute node. Though the iterative gVCF genotyper may attempt to create shards of equal size, the shards may be of unequal sizes. For example, the shards may be prevented from spanning chromosomes, which may result in the shards ending with chromosomes in unequal shard sizes. Each shard may be of equal size, except at the end of a chromosome (e.g., the residual), which may be smaller than the equal size.

Each core processor that is accessible by the compute nodes or virtual machines may have specific threading. Each core may have specific threading. A variable number of software threads may be implemented. One or more threads may be implemented for each CPU core. For example, a single thread may be implemented by each CPU core. The number of threads implemented by each CPU core may be changed in response to user input. Each thread of the iterative gVCF genotyper may be assigned to extract, at 810, sequencing data from a respective gVCF file or portion of the gVCF file to create respective cohort and census files for respective gVCF file or portion of the gVCF file. The iterative gVCF genotyper may split the cohort and/or census file generation of each of the batches 802, 804, 806 into respective shards such that each of the shard generated cohort and census files is generated by a separate shard. Each of the census files generated by the shards processing a common batch of samples may be aggregated at 820 into a batch census file. The batch census file may include similar fields as the global census file, including the region, RAGT statistics, and optionally site statistics. The batch census files may differ from the global census file in sample size, as the batch census file may include census data for a given batch of gVCF files and the global census file may include census data from multiple batches of samples.

The census files created from the shards processing the gVCF files in batch 802 may be aggregated to create a first batch census file for batch 802, the census files created from the shards processing the gVCF files in batch 804 may be aggregated to create a second batch census file for batch 804, and the census file created from the shards processing the gVCF files in batch 806 may be aggregated to create a third batch census file for batch 806. Each of the batch census files may aggregated at to generate a global census file.

The batch census file RAGT statistics may be calculated from the batch cohort file (e.g., by taking the sample count per metrics value). The global census file may include the RAGT statistics aggregated from the RAGT statistics in each per batch census file by summing up the sample count per metrics value. The global census file may include variant and hom-ref metrics for each of the samples in the cohort files. The global census file may also include this information for each genomic position. The batch census files and the global census file may include a site statistics field that includes the variant calling data, e.g., reference allele and alternate allele normalization, alternate allele ordering and remapping of index between common alternate alleles, and alternate allele in each RAGT case.

As each shard may be implemented to generate cohort data for a respective gVCF file or portion of the gVCF file, the shards may each use their respective cohort file in a batch to generate portions of a multi-sample VCF file. For example, each shard that has been implemented to process the respective gVCF file or portion of the gVCF file in the batch 802 to generate a corresponding cohort file may use their respective cohort file and the global census file to create a portion of the msVCF 842. Each shard that has been implemented to process the respective gVCF file or portion of the gVCF file in the batch 804 to generate a corresponding cohort file may use their respective cohort file and the global census file to create a portion of the msVCF 844. Each shard that has been implemented to process the respective gVCF file or portion of the gVCF file in the batch 806 to generate a corresponding cohort file may use their respective cohort file and the global census file to create a portion of the msVCF 846. Thus, as illustrated at 830 in the process 800, each msVCF 842, 844, 846 may be generated on a per-shard, per-batch basis. When each sample is assigned to a shard, samples may be freely grouped in a defined group (e.g., such as a subpopulation or case/control group(s)). When each sample is assigned to a shard, the shard may handle (e.g., only handle) one sample, which may allow for efficient compression compared to cohorts of multiple samples.

Each compute node or virtual machine may perform at least two levels of parallelization for processing, aggregating, and/or generating data for a corresponding region of the sequence data. Each compute node or virtual machine may process a specific region of the data in the gVCF files in a given batch.

The iterative gVCF genotyper may include runtime option parameters for implementing the process 800. The runtime option parameters may set the number of regions to split the genome in order to parallelize the process on distributed nodes. As each core may have specific threading, the runtime option parameters may also set the number of subregions to split one region to run on different threads on the same node. The runtime option parameters may set the data buffer size (e.g., to further split the subregion) so that each thread does not utilize an excessive amount of memory at any point of time.

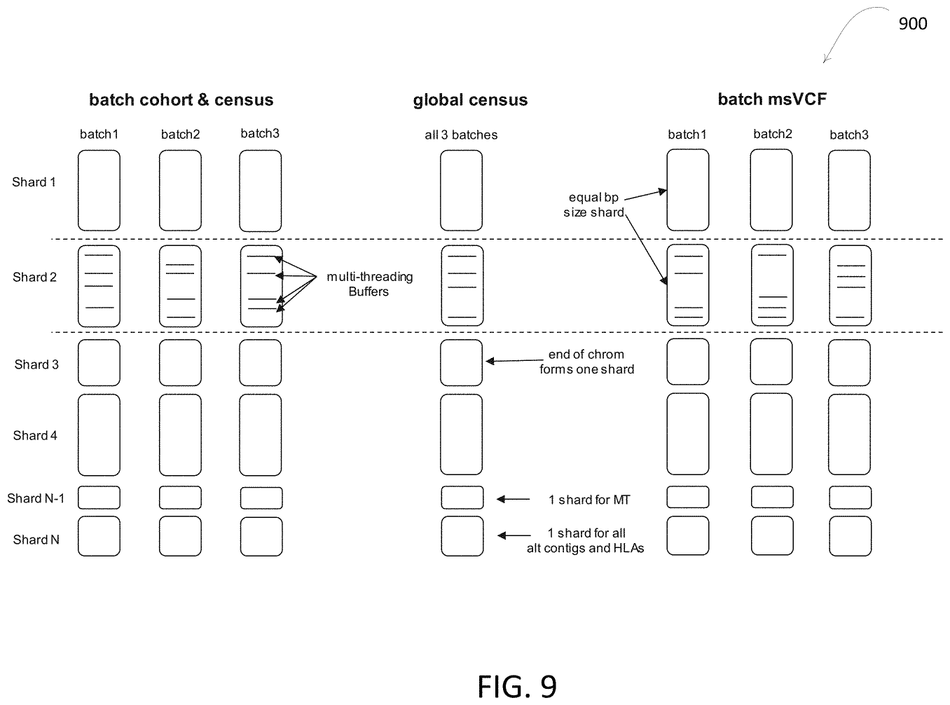

FIG. 9 depicts an example process 900 incorporating parallel processing across batches of sample data. In an example, the iterative gVCF genotyper may assign processing resources to enable the parallel processing being described. The illustration shown in FIG. 9 shows each batch being processed by multiple shards. In the example process 900, each shard may be assigned a specific portion of the cohort and census files for each batch. For example, a shard (e.g., shard 1) may process its portion of the cohort and census files for batch 1 and then proceed to its portion of the cohort and census files for batch 2 upon completion of processing its portion of the cohort and census files for batch 1. The shard may then proceed to its portion of the cohort and census files for batch 3 upon completion of processing its portion of the cohort and census files for batch 2. Each shard may have a uniform definition, for example, such that the same genomic shard from different batches can be aggregated (e.g., without mismatch in shard boundaries). Each shard may leverage multiple threads for processing the portions of each batch. Within each shard, the thread level splitting (or subregion) can be adjusted differently from batch to batch. The thread level splitting may be adjusted differently from batch to batch because the output file (e.g., cohort or census) of a multithread process is concatenated (e.g., always concatenated) into one output file (e.g., cohort or census) per shard.

In the example process 900, each shard may be assigned a specific portion of the global census file for being generated by the shard. For example, a shard (e.g., shard 1) may process its portion of the global census file upon completion of its portions of the batch cohort and census files. An end of chromosome may be used to make the end of one or more shards. For example, one shard may be assigned for mitochondrial (MT) genome data and/or one shard may be assigned for alternate configurations and Human Leukocyte Antigens (HLAs). These additional shards (e.g., mitochondrial and alternate contigs including HLA) may be present in the human reference genome. They may be kept in separate shards because the ploidy may be different from autosomes and sex chromosomes. The size of these contigs may be relatively small, and may not implement further parallelization by more shards.

In the example process 900, each shard may be assigned a specific portion of each msVCF file. For example, a shard (e.g., shard 1) may process its portion of the msVCF file for batch 1 and then proceed to its portion of the msVCF file for batch 2 upon completion of processing its portion of the msVCF file for batch 1. The shard may utilize its portion of the batch file corresponding to the msVCF file being generated and its portion of the global census file to generate the shard-specific portion of the msVCF file for each batch. The shard may then proceed to its portion of the msVCF file for batch 3 upon completion of processing its portion of the msVCF file for batch 2.

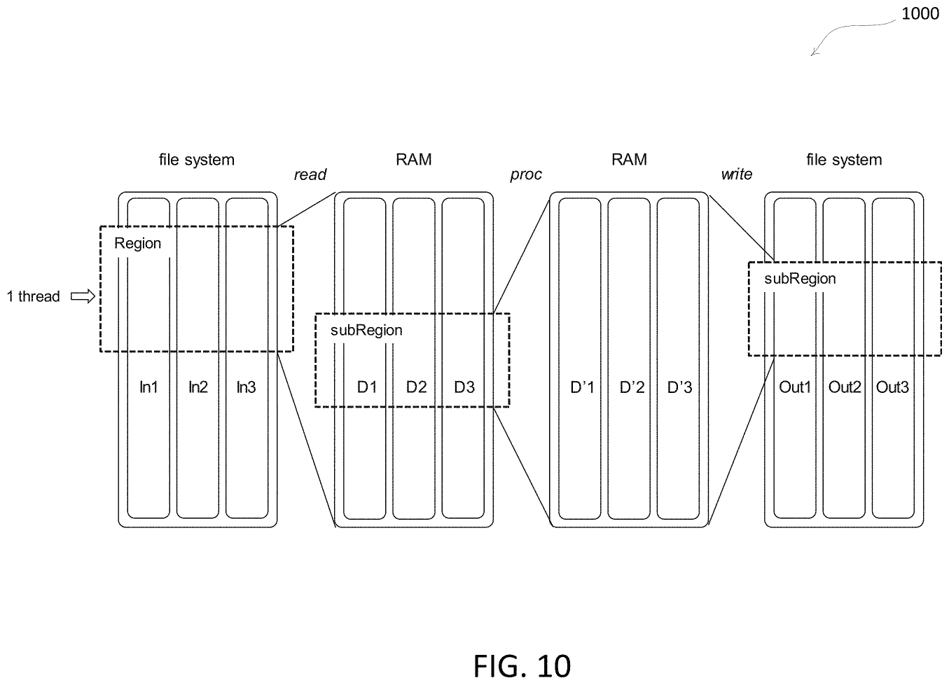

FIG. 10 depicts an example Genome Data Operator design 1000. The example Genome Data Operator design 1000 may be used to read, process, and/or write genomic data. The region and/or subregion sizes may be configurable, for example, depending on per thread ram size and/or type and number of input files and in-memory data structure size. The example Genome Operator design 1000 may be used in the processing of batches of sample data from a sequencing device for generating one or more msVCF files.

FIG. 10 shows a parallelization that may be performed on the same compute node (e.g., where a shard is already fixed). Given the region as defined by one genomic shard, and the number of threads on a given compute node, the analysis may be further parallelized by concurrent thread by splitting the shard into per thread regions. When each thread process one such region, a portion of the data in the region may be loaded from the file system. For example, each thread may process records in one subregion at a time. The subregion size may be configured to maximize the usage of memory per thread and/or avoid overloading the total memory of the system when each of the threads are fully loaded. Each thread may process the subregions contained in a region in serial. The threads may process regions contained in a shard in parallel.

In generating the global census files and msVCF files as described herein, the global census files and msVCF files themselves may grow in size as global census data is aggregated from different batches of sample data and updated in the global census files and/or included in the msVCF files. The global census data may have stored therein a representation of the alleles that are represented in a field (e.g., RAGT field) of the gVCF files from which data is being aggregated from multiple sites. The size of the global census data in the global census file and/or the msVCF files may be reduced by reducing duplicative alleles that may be represented in differently in the global census data. The reduction in the size of these files would allow for reduced memory and processing requirements when storing and analyzing the global census data.

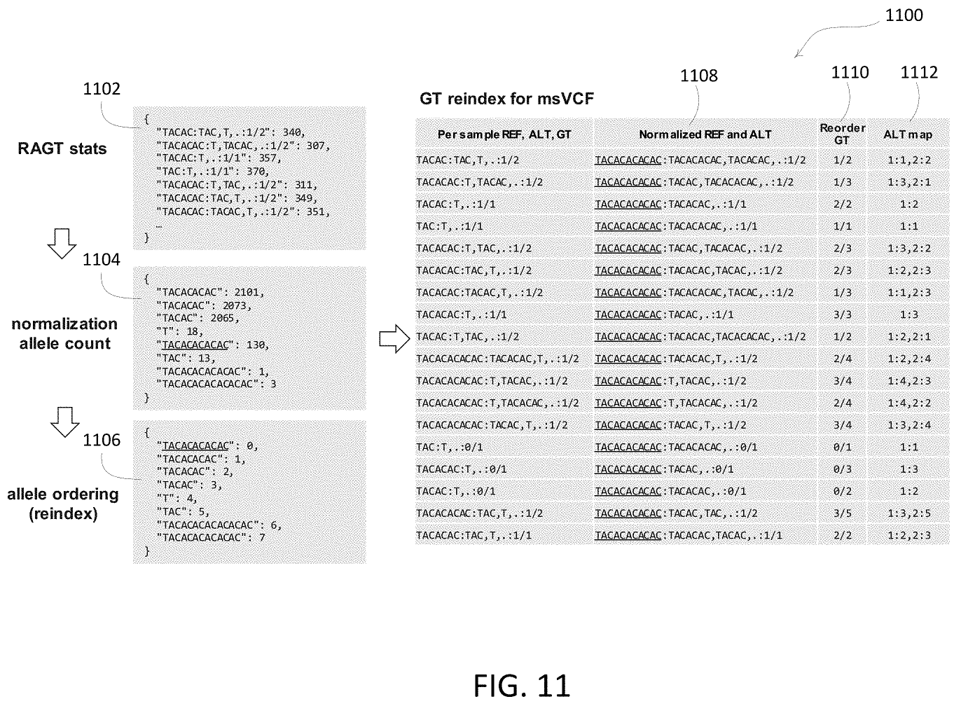

One way to reduce the representation of duplicative alleles would be to normalize each allele based on a reference allele. This normalization may also allow for a more accurate allele count for the possible variances that may be discovered for a cohort. FIG. 11 depicts an example process 1100 that may be implemented by one or more computing devices (e.g., such as the server device 102, the client device 108, and/or the sequencing device 114 shown in FIG. 1) for normalizing and reindexing a genotype for an msVCF file. Though the process 1100 shows normalization and reindexing techniques that may be implemented on an msVCF file, similar normalization and reindexing techniques may be implemented for a global census file. The example process 1100, or portions thereof, may use RAGT statistics to normalize population level Reference and Alternate alleles, order the population level Alternate alleles, reindex sample level genotype, and store the data in a census file and msVCF file. The Reference and Alternate alleles and GT reindex and ALT index mapping generation in FIG. 11 may be performed during census aggregation and/or gVCF aggregation resulting in site statistics. The site statistics may be stored in a site statistics field of the census file. The site statistics may be inserted in the msVCF file (e.g., in a site statistics field) during generation. The process 1100, or portions thereof, may be performed to efficiently store genome variant data in an msVCF file and/or global census file. The process 1100 may be implemented by an iterative gVCF genotyper being executed on one or more processors of the one or more computing devices. The process 1100, or portions thereof, may be performed at a single computing device or may be distributed across multiple computing devices (e.g., multiple server(s), sequencing device(s) and/or client computing device(s)). The process 1100, or portions thereof, may reduce the amount of processing resources and/or storage space (e.g., memory) used by the computing device(s) during a variant analysis.

The example process 1100 may begin when the processor executing the iterative gVCF genotyper receives, at 1102, RAGT statistics in gVCF files. One or more of the gVCF files may have been recently received by the iterative gVCF genotyper. Each of the gVCF files may be associated with a respective sample of a plurality of samples. Additionally or alternatively, one or more of the gVCF files may have been previously received and stored at the one or more computing devices and/or a database (e.g., such as the database 116 shown in FIG. 1). The RAGT statistics may be associated with the plurality of samples. For example, the RAGT statistics may be stored on the one or more computing devices and/or in a database (e.g., such as database 116 shown in FIG. 1) and the process 1100 may be used to update the genotype reindex, reference allele, and/or alternate allele mapping based on the received (e.g., new) RAGT statistics. The processor executing the iterative gVCF genotyper may identify a plurality of reference alleles and a plurality of alternate alleles associated with the plurality of samples using the RAGT statistics.

The iterative gVCF genotyper may sum, at 1104, the instances (e.g., occurrence) of each unique allele (e.g., the plurality of reference alleles and plurality of alternate alleles) into an allele count, for example, based on the RAGT statistics. For example, the one or more computing devices may determine how many instances of each unique allele (e.g., reference alleles and alternate alleles) are present in the RAGT statistics. It will be appreciated that although the example shown in FIG. 11 includes 8 unique alleles, the example process 1100 is not limited to this number of alleles. Rather, the example process 1100 may be used when the number of unique alleles is greater than or less than 8.

The iterative gVCF genotyper may order at, 1106, the allele counts, for example, based on length. The allele order determined at 1106 may begin with the reference allele counts and end with the alternate allele counts. Stated differently, the reference allele counts may be listed before the alternate allele counts in the allele order. For example, the longest reference allele may be listed first in the allele order and the remaining reference alleles may be listed in order of decreasing length. Each of the ordered allele counts may be assigned a number between 0 and the total number of unique alleles (e.g., such as 7 in the example shown in FIG. 11) in the RAGT statistics. Zero (0) may be assigned to the longest reference allele and the remaining alleles may be ordered based on occurrence. For example, the alleles may be listed in descending order by occurrence in the RAGT statistics. It will be appreciated that the alleles are not limited to being ordered in descending order by occurrence, instead the alleles may be ordered in other ways, for example, by descending order of length, etc.

At 1108, the alleles may be normalized, for example, based on the longest reference allele. For example, the iterative gVCF genotyper may select a normalized reference allele. The normalized reference allele may be the longest reference allele. The alleles for each sample in the RAGT statistics may be normalized by being extended the alleles to the length of the longest reference allele. The alternate alleles of each reference allele may also be extended by the same amount (e.g., number of bases) as the corresponding reference allele. For example, when a reference allele is extended by 2 (e.g., two base pairs or a nucleotide), each alternate allele of the reference allele is also extended by 2 (e.g., two bases). A normalized representation of each sample may be generated using the normalized reference allele such that each of the plurality of alternate alleles are indexed using the normalized reference allele.

The normalization of the alternate alleles may allow for alternate alleles having the same reference to be put in the same line of the data. The same reference allele and alternate allele may be grouped by the same reference alleles. In order to allow for the normalized reference alleles and normalized alternate alleles to be included in the same line,

At 1110, the genotype for each sample is reordered, for example, based on the normalized reference and/or alternate alleles. For example, assume the original genotype in the RAGT field is 1/2, and the original reference allele is TACAC, the original ALT alleles are TAC, T. After renormalization, if the common reference allele is TACACACACAC (added ACACAC), then the normalized alternate allele is TACACACAC, TACACAC. According to the order of each of the normalized reference and alternate alleles, if TACACACAC allele has index 3 and TACACAC index 5, then the ordered genotype is 3/5 (instead of 1/2), and the alternate allele mapping (from old to new) 1=>3, 2=>5. After the allele reordering, at 1112, there may be generated an alternate allele mapping for each sample, for example, based on the normalized reference and/or alternate alleles. The common reference allele may refer to a common (renormalized) reference allele shared among each of the samples at a particular site. The reordering, at 1112, may be implemented because different samples may have different alternate alleles at the same genomic position. In order to write into an msVCF, you may enforce an ordering which is consistent across positions. This re-ordering the genotype fields may be implemented because the genotype may refer to indices in the list of alternate alleles.

The normalized reference and alternate alleles, the reordered genotype, and/or the alternate mapping may be stored in the site statistics field of the census file that includes the variant calling data, e.g., reference allele and alternate allele normalization, alternate allele ordering and remapping of index between common alternate alleles, and alternate alleles in each RAGT case. An example site statistics field of a batch with 12 samples may be represented by the following,

```
{
  “REF”: “TACACAC”,
  “HAS_VAR”: true,
  “NS_GT”: 12,
  “NS_NOGT”: 0,
  “NS_NODATA”: 0,
  “ALT”: “TACAC,TAC,T,X”,
  “AC”: {
    “TACAC”: 10,
    “TAC”: 9,
    “T”: 5
  },
  “ALT_MAP”: {
    “TACAC:TAC,T,X:1/2”: {
      “1”: 1,
      “2”: 2,
      “3”: 4
    },
    “TACACAC:T,TACAC,X:1/2”: {
      “1”: 3,
      “2”: 1,
      “3”: 4
    },
    “TACAC:T,X:1/1”: {
      “1”: 2,
      “2”: 4
    },
    “TAC:T,X:1/1”: {
      “1”: 1,
      “2”: 4
    },
    “TACACAC:T,TAC,X:1/2”: {
      “1”: 3,
      “2”: 2,
      “3”: 4
    },
    “TACACAC:TAC,T,X:1/2”: {
      “1”: 2,
      “2”: 3,
      “3”: 4
    },
    “TACACAC:TACAC,T,X:1/2”: {
      “1”: 1,
      “2”: 3,
      “3”: 4
    }
  }
}
```

The site statistics field may comprise the RAGT statistics for a batch. The site statistics field may include a number count for the value of each census field. The number count may be used for the population genome. The site statistics field may include key census file information that is used as input to msVCF file.

The normalized reference and alleles, reordered genotype, and alternate mapping may be used to generate a msVCF file for the plurality of samples associated with the RAGT statistics. For example, the normalized reference and alleles, reordered genotype, and alternate mapping may be output to the msVCF file. The msVCF file may include the common reference allele in a REF column. The generated msVCF file may be stored by the iterative gVCF genotyper on one or more computing devices and/or the database.

The process 1100 may be repeated as additional gVCF file(s) are received. The RAGT statistics from the additional gVCF file(s) may be added to the previously received RAGT statistics. For example, the iterative gVCF genotyper may receive the additional gVCF file(s) associated with one or more additional samples. The iterative gVCF genotyper may identify one or more reference alleles and one or more alternate alleles associated with the one or more additional samples. The one or more reference alleles and the one or more alternate alleles may be added to the allele count. The one or more computing devices may determine whether any of the one or more reference alleles are longer than the longest reference allele. When a reference allele (e.g., in the additional gVCF file(s)) is longer than the previous longest reference allele, the iterative gVCF genotyper may select the reference allele as an updated longest reference allele. The plurality of reference alleles and the plurality of alternate alleles may be normalized by extending to correspond to the length of the updated longest reference allele. The genotype ordering and the alternate mapping may be updated based on the information received in the additional gVCF file(s). As this data may be updated, the global census file may be updated and another msVCF file may be generated for each batch of data that is received.

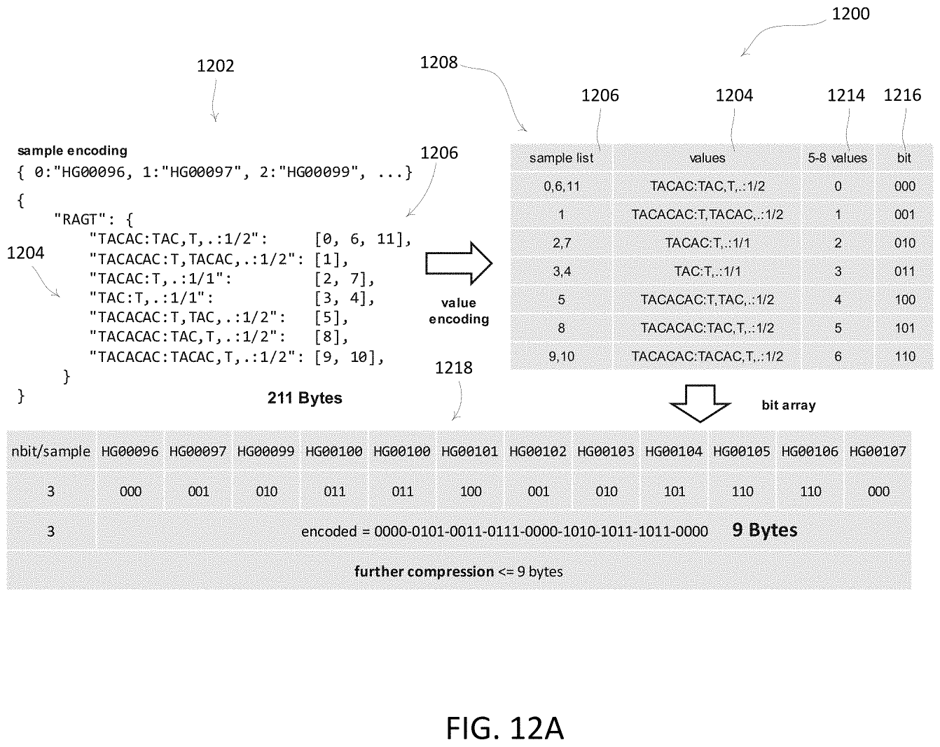

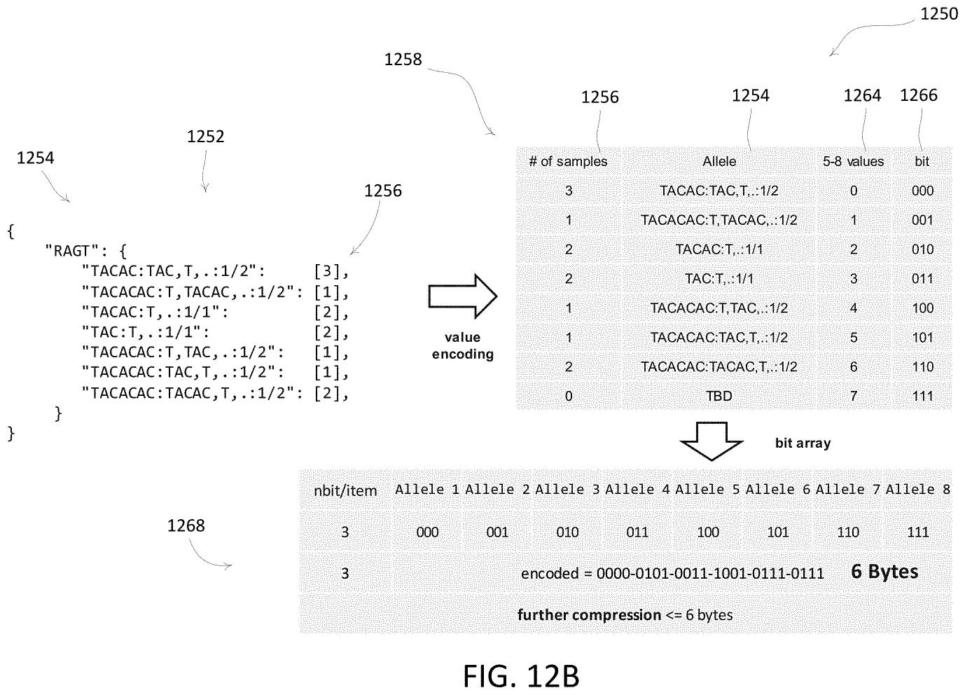

Example embodiments are described herein for generating cohort files and census files that have a smaller file size than the gVCF files comprising the sample data on which the cohort files and census files are generated. The size of the cohort files and census files may be further reduced using one or more compression and/or serialization techniques described herein. FIG. 12A depicts an example process 1200 that may be implemented by one or more computing devices (e.g., such as the server device 102, the client device 108, and/or the sequencing device 114 shown in FIG. 1) to encode cohort and/or census data in a compressed format. The process 1200, or portions thereof, may be performed to efficiently store variant data in a census file, a cohort file, and/or a msVCF file. The process 1200, or portions thereof, may be implemented by an iterative gVCF genotyper being executed on one or more processors of the one or more computing devices. The process 1200, or portions thereof, may be performed at a single computing device or may be distributed across multiple computing devices (e.g., multiple server(s), sequencing device(s) and/or client computing device(s)). The process 1200, or portions thereof, may reduce the amount of processing resources and/or storage space (e.g., memory) used by the computing device(s) during a variant analysis.

The example process 1200 may begin after the iterative gVCF genotyper generates the cohort file and/or census file for being compressed. As further described herein, the fields in the cohort file and the census file may each include a subset of the fields included in the gVCF files. Each of the fields may include genome variant data and/or other genotype data that is associated with a batch of gVCF files. In the example, provided in FIG. 12A, the compression process 1200 is provided for compressing the allele data for the RAGT field in a cohort file 1202. However, similar types of compression may be performed for the cohort data and/or census data for each field of the corresponding cohort files and/or census files, and will be further described elsewhere herein.

As described in the example process 1200, the iterative gVCF genotyper may encode the data in one or more fields of the cohort file, such that the data may be compressed into a bit array 1218. As described herein, each field in the cohort file may include a series of unique values or metrics for the field that are each followed by an integer array of the gVCF files or samples that include the unique value or metric. As shown in FIG. 12A, the RAGT field in the cohort file 1202 may include a series of unique values or metrics 1204 previously identified in the batch of samples from which the cohort file was generated. The unique values or metrics 1204 may be related to one or more fields in the RAGT statistics, such as reference alleles, alternate alleles, and/or genotype for each of the samples associated with the variant data, for example. Each unique value or metric 1204 may be followed by an integer array that identifies the one or more samples or gVCF files 1206 having the preceding unique value or metric 1204.

The iterative gVCF genotyper may encode the data in the RAGT field using a bitmap 1208, such that the unique values 1204 and the corresponding samples or gVCF files may be represented as a series of bits. To generate the bitmap 1208, the iterative gVCF genotyper may identify the total the number of unique values or metrics 1204 in the RAGT field. The iterative gVCF genotyper may identify a binary value length of the number of bits 1216 that may be implemented to represent the total number of unique values or metrics 1204. For example, the total number of unique values or metrics 1204 in the RAGT field of the cohort file 1202 is seven. The iterative gVCF genotyper may identify the lowest number of bits that may be implemented to represent the seven unique values in the RAGT field of the cohort file 1202. As a bit sequence of three bits is the lowest number of bits that may be implemented to represent the unique values in the RAGT field, the bits 1216 may be set to a three-bit sequence. It would be understood that another binary value length for the number of bits 1216 may be implemented to represent other numbers of unique values or metrics.

With regard to the RAGT field, the iterative gVCF genotyper may identify a plurality of reference alleles and/or a plurality of alternate alleles, for example, using the RAGT statistics in the plurality of gVCF files. The iterative gVCF genotyper may determine which of the plurality of samples have common reference and alternate alleles. The samples may be distributed into allele groups. Each of the allele groups may comprise one or more samples with common reference and alternate alleles. For example, when two samples are found to have common (e.g., the same) reference and alternate alleles, the two samples may be grouped together into an allele group. Each allele group may be included in the unique values or metrics 1204. The allele groups may be aggregated into a list of allele groups represented in the unique values or metrics 1204. The iterative gVCF genotyper may determine the number of bits 1216 based on the number of allele groups (e.g., values). For example, the iterative gVCF genotyper may select a binary value length based on the number of allele groups.

The bitmap 1208 may include the unique values or metrics 1204 and the corresponding identifiers of the samples or gVCF files 1206 having the unique values or metrics 1204. Each of the values or metrics 1204 and the corresponding identifiers of the samples or gVCF files 1206 may be listed in the same row in the bitmap 1208 as the representative bits 1216 corresponding thereto. In the bitmap 1208, the bits 1216 are incremented with each row in the bitmap 1208, though other implementations would be understood. The integer values 1214 identify the integer value of the bits 1216 in each row.

The iterative gVCF genotyper may generate a bit array 1218 for the plurality of gVCF files by aggregating the unique assigned binary values (e.g., the unique values or metrices 1204). The bitmap 1208 may be used by the iterative gVCF genotyper for encoding the data in the RAGT field of the cohort file 120 into the bit array 1218 and for decoding the bit array 1218 to generate the data in the RAGT field of the cohort file 1202. The iterative gVCF genotyper may encode the bit array 1218 based on the order of the associated samples in the plurality of gVCF files. For example, the iterative gVCF genotyper may generate a bit sequence that comprises a series of 3-bit sequences that represent the values 1204 of the samples 1206 in numerical order. As shown in FIG. 12A, the bit array 1218 may start with the bits 1216 representing the RAGT value 1204 for sample zero (e.g., “000”). The iterative gVCF genotyper may append the bits 1216 representing the RAGT value 1204 for sample one (e.g., “0001”), and so on. The iterative gVCF genotyper may store the bit array 1218.

The bitmap may be different between cohort files and census files. The bitmap may be generated per record and per metrics field, as the bitmap may depend on the number of unique metrics values and the sample identifiers. In the cohort file, the bitmap may encode the per sample hashed value. In an example using the GQ field, sample 1 value 10 may be hashed to 0, sample 2 value 20 may be hashed to 1, and sample 3 value 10 may be hashed to 0. The bitmap may encode the array [0,1,0] in the order of sample identifier with value string “10,20” (two unique values). In the census file, the bitmap may encode the sample count per metrics value. In another example using the GQ field, 25 samples having the value 10 and 36 samples having the value 20, the bitmap may encode the array [25,36] in the order of value string “10,20”.

In the example shown in FIG. 12A, the process 1200 may compress an input of 211 bytes to an output of 9 bytes. The example process 1200 shows an example to compress metrics for a field (e.g., RAGT) with 7 unique values, which can be compressed using 3 bits (e.g., from 000 to 110) to represent these 7 values. The number of bits changes depending on the number of unique values to compress, e.g. 2 unique values=1 bit, 256 unique values=8 bits. So, the compression may vary from record to record, from metrics to metrics. The data being compressed may be different between cohort and census. The example process 1200 may be used by the iterative gVCF genotyper to compress one or more predefined fields in the gVCF files described herein. For example, the predefined fields may be compressed when storing the data in cohort and census files.

It will be appreciated that although the process 1200 is shown in FIG. 12A performing compression on RAGT statistics, the process 1200 is not limited to compression of RAGT statistics. For example, the process 1200 may be used to compress data in other fields of the gVCF files. It will also be appreciated that although the process 1200 is shown and described with reference to FIG. 12A as compressing a data in a cohort file, a similar process may be implemented to compress data in a census file. It will be further appreciated that although the process 1200 is shown and described as being implemented to compress sequencing data, the process 1200 is not limited to compression of sequencing data. Instead, the process 1200 may be used to compress other types of data.

FIG. 12B depicts an example process 1250 that may be implemented by one or more computing devices (e.g., such as the server device 102, the client device 108, and/or the sequencing device 114 shown in FIG. 1) to encode cohort and/or census data in a compressed format. The process 1250, or portions thereof, may be performed to efficiently store variant data in a census file, a cohort file, and/or a msVCF file. The process 1250, or portions thereof, may be implemented by an iterative gVCF genotyper being executed on one or more processors of the one or more computing devices. The process 1250, or portions thereof, may be performed at a single computing device or may be distributed across multiple computing devices (e.g., multiple server(s), sequencing device(s) and/or client computing device(s)). The process 1250, or portions thereof, may reduce the amount of processing resources and/or storage space (e.g., memory) used by the computing device(s) during a variant analysis.

The example process 1250 may begin after the iterative gVCF genotyper generates the cohort file and/or census file for being compressed. As further described herein, the fields in the cohort file and the census file may each include a subset of the fields included in the gVCF files. Each of the fields may include genome variant data and/or other genotype data that is associated with a batch of gVCF files. In the example, provided in FIG. 12B, the compression process 1250 is provided for compressing the allele data for the RAGT field in a census file 1252. However, similar types of compression may be performed for the cohort data and/or census data for each field of the corresponding cohort files and/or census files, and will be further described elsewhere herein.

As described in the example process 1250, the iterative gVCF genotyper may encode the data in one or more fields of the census file, such that the data may be compressed into a bit array 1268. As described herein, each field in the census file may include a series of unique values or metrics for the field that are each followed by a sample count of the gVCF files or samples that include the unique value or metric. As shown in FIG. 12B, the RAGT field in the census file 1252 may include a series of unique values or metrics 1254 (e.g., such as the alleles shown in FIG. 12B) previously identified in the batch of samples from which the census file was generated. The unique values or metrics 1254 may be related to one or more fields in the RAGT statistics, such as reference alleles, alternate alleles, and/or genotype for each of the samples associated with the variant data, for example. Each unique value or metric 1254 may be followed by a sample count 1256 that indicates how many of the one or more samples or gVCF files 1252 have the preceding unique value or metric 1254.

The iterative gVCF genotyper may encode the data in the RAGT field using a bitmap 1258, such that the unique values 1254 and the sample counts 1256 may be represented as a series of bits. To generate the bitmap 1258, the iterative gVCF genotyper may identify the total number of unique values or metrics 1254 in the RAGT field of the census file. The iterative gVCF genotyper may identify a binary value length of the number of bits 1266 that may be implemented to represent the total number of unique values or metrics 1254. The iterative gVCF genotyper may identify the lowest number of bits that may be implemented to represent the unique values (e.g., unique alleles) in the RAGT field of the census file 1252. As a bit sequence of three bits is the lowest number of bits that may be implemented to represent the unique values in the RAGT field, the bits 1266 may be set to a three-bit sequence. It should be understood that another binary value length for the number of bits 1266 may be implemented to represent other numbers of unique values or metrics.

With regard to the RAGT field, the iterative gVCF genotyper may identify a sample count of the alleles using the RAGT statistics in the plurality of gVCF files. For example, when two samples are found to have common (e.g., the same) reference and alternate alleles, the sample count for that allele (e.g., allele group) may be listed in the sample count 1256. The allele groups may be aggregated into a list of allele groups represented in the sample count 1256. The iterative gVCF genotyper may determine the number of bits 1266 based on the number of allele groups (e.g., values). For example, the iterative gVCF genotyper may select a binary value length based on the number of alleles/allele groups

The bitmap 1258 may include the unique values or metrics 1254 and the sample counts of the samples or gVCF files 1256 having the unique values or metrics 1254. Each of the values or metrics 1254 and the sample counts of the samples or gVCF files 1256 may be listed in the same row in the bitmap 1258 as the representative bits 1266 corresponding thereto. In the bitmap 1258, the bits 1266 are incremented with each row in the bitmap 1258, though other implementations should be understood. The integer values 1264 identify the integer value of the bits 1266 in each row.

The iterative gVCF genotyper may generate a bit array 1268 for the plurality of gVCF files by aggregating the unique assigned binary values (e.g., the unique values or metrices 1254). The bitmap 1258 may be used by the iterative gVCF genotyper for encoding the data in the RAGT field of the census file 1252 into a bit array 1268 and for decoding the bit array 1268 to generate the data in the RAGT field of the census file 1252. The iterative gVCF genotyper may encode the bit array 1268 based on the order of the associated samples in the plurality of gVCF files. For example, the iterative gVCF genotyper may generate a bit sequence that comprises a series of 3-bit sequences that represent the sample counts 1256 of the alleles/allele groups 1254 in numerical order. As shown in FIG. 12B, the bit array 1268 may start with the bits 1266 representing the sample count 1256 for Allele 1 (e.g., TACAC:TAC,T, . :1/2). The iterative gVCF genotyper may append the bits 1266 representing the sample count 1256 for Allele 2 (e.g., TACACAC:T, TACAC, .:12), and so on. The iterative gVCF genotyper may store the bit array 1268.

The bitmap 1258 may be generated per record and per metrics field, as the bitmap may depend on the number of unique metrics values and the sample identifiers. In the census file, the bitmap may encode the sample counts. In an example using the GQ field, 25 samples having the value 10 and 36 samples having the value 20, the bitmap may encode the array [25,36] in the order of value string “10,20”.

In the example shown in FIG. 12B, the process 1250 may compress an input of 211 bytes to an output of 6 bytes. The example process 1250 shows an example to compress metrics for a field (e.g., RAGT) with 7 unique values, which can be compressed using 3 bits (e.g., from 000 to 110) to represent these 7 values. The number of bits changes depending on the number of unique values to compress, e.g. 2 unique values=1 bit, 256 unique values=8 bits. So, the compression may vary from record to record, from metrics to metrics. The data being compressed may be different between cohort and census. The example process 1250 may be used by the iterative gVCF genotyper to compress one or more predefined fields in the gVCF files described herein. For example, the predefined fields may be compressed when storing the data in cohort and census files.

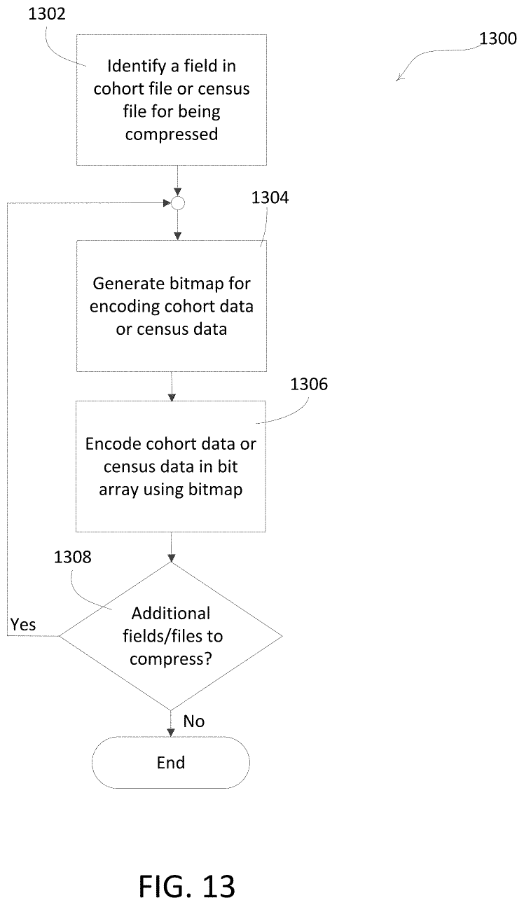

FIG. 13 is a flow diagram depicting an example method 1300 that may be implemented by one or more computing devices (e.g., such as the server device 102, the client device 108, and/or the sequencing device 114 shown in FIG. 1) to perform compression of cohort and/or census data. The method 1300, or portions thereof, may be performed by an iterative gVCF genotyper to efficiently store genome variant data and/or other genome data in a census file, a cohort file, and/or a msVCF file. The method 1300, or portions thereof, may be performed at a single computing device or may be distributed across multiple computing devices (e.g., multiple server(s), sequencing device(s) and/or client computing device(s)). The method 1300, or portions thereof, may reduce the amount of processing resources and/or storage space (e.g., memory) used by the computing device(s) during a variant analysis.

As shown in FIG. 13, the iterative gVCF genotyper may identify a field in the cohort file and/or the census file for being compressed at 1302. The fields in the cohort file and/or the census file may each include a subset of the fields included in the gVCF files. Each of the fields may include genome variant data and/or other genotype data that is associated with a batch of gVCF files. The fields may be formatted as text fields. As described herein, each field in the cohort file may include a series of unique values or metrics for the field that are each followed by an integer array of the gVCF files or samples that include the unique value or metric. As also described herein, each field in the census file may include a series of unique values or metrics for the field that are each followed by a sample count for the number of samples having the unique value or metric in the batch of samples represented by the census file.

At 1304, the iterative gVCF genotyper may generate a bitmap for encoding the cohort data or the census data for the identified field. To generate the bitmap, the iterative gVCF genotyper may identify the total the number of unique values or metrics in the field. The iterative gVCF genotyper may identify a binary value length for the number of bits that may be implemented to represent the total number of unique values or metrics in the field of the cohort file or the census file. For example, the iterative gVCF genotyper may identify and select the lowest number of bits that may be implemented to represent the total number of unique values or metrics in the field of the cohort file or the census file in the bitmap.

For the cohort file, the bitmap may include the unique values or metrics for the field and the corresponding identifiers of the samples or gVCF files having the unique values or metrics. For the census file, the bitmap may include the unique values or metrics for the field and a corresponding sample count for the number of samples having the unique value or metric in the batch of samples represented by the census file. Each of the values or metrics may be listed in the same row in the bitmap as the representative bits corresponding thereto.

At 1306, the iterative gVCF genotyper may encode the cohort data or census data for the identified field in a bit array using the bitmap. The bitmap may be used by the iterative gVCF genotyper for encoding the data in the field of the cohort file or the census file into a bit array. The iterative gVCF genotyper may encode the bit array for the cohort data based on the order of the associated samples in the plurality of gVCF files, such that the bits representing the unique values or metrics are encoded in an increasing numeric order by sample or gVCF identifier. The census data in the census file may be ordered based on the metrics value of each field. For metrics A, if the values are v1, v2, v3 and count c1, c2, c3, then the counts [c1, c2, c3] are bit compressed, and the number of bits depend on the max value among c1, c2, c3.

At 1308, the iterative gVCF genotyper may determine whether additional fields in the cohort file or census file are to be compressed, or whether additional cohort files or census files are to be compressed. If there are additional fields or files to compress, the method 1300 may return to 1304. If there are not additional fields or files to compress, the method 1300 may end.

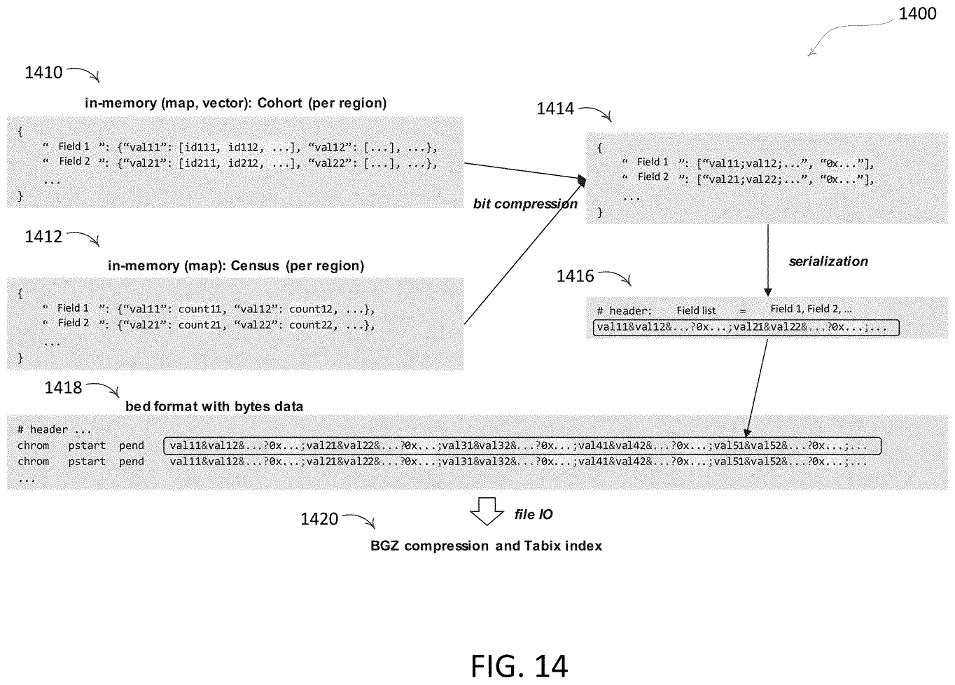

The compressed cohort data and the compressed census data may be further compressed and serialized, as illustrated in FIG. 14. FIG. 14 depicts an example process 1400 that may be implemented by one or more computing devices (e.g., such as the server device 102, the client device 108, and/or the sequencing device 114 shown in FIG. 1) for data serialization and compression. The process 1400, or portions thereof, may be performed by an iterative gVCF genotyper. In the example process 1400, cohort data and census data may be serialized into binary data using bit compression and may be further encoded into ascii data such that the data (together with the region information) may be compressed using bgzip compression algorithm and can be indexed by a tabix algorithm, which allow for genomic region querying. bgzip and tabix are public algorithms implemented in HTSlib. The process 1400, or portions thereof, may be performed to efficiently store genome variant data and/or other genotype data in a census file, a cohort file, and/or a msVCF file. The process 1400, or portions thereof, may be performed at a single computing device or may be distributed across multiple computing devices (e.g., multiple server(s), sequencing device(s) and/or client computing device(s)). The process 1400, or portions thereof, may reduce the amount of processing resources and/or storage space (e.g., memory) used by the computing device(s) during a variant analysis.

The process 1400 may use a binary data compression scheme (e.g., such as the process 1200 shown in FIG. 12) to directly encode gVCF data into binary data in unit of bits (instead of serialized data in ascii). The process 1400 may implement an alternative compression algorithm (e.g., lz4 instead of bgzip) and customized indexing algorithm (e.g., instead of tabix), for example, to further improve compression and/or query efficiency. Additionally, or alternatively, the example process 1400 may incorporate one or more other methods of compression.

Cohort data 1410 (e.g., in one or more cohort files) may be received at the iterative gVCF genotyper. Census data 1412 (e.g., in one or more census files and/or a global census file) may be received at the iterative gVCF genotyper. The cohort data and/or census data may be separated by region. Bit compression may be performed on the cohort and census data to generate a compressed file 1414. For example, the cohort and census data may be represented (e.g., using a similar process to the process 1200 shown in FIG. 12) using a bit sequence (e.g., a binary bit sequence). The cohort or census file headers comprising the field values may be serialized in the serialized data file 1416. The cohort or census file headers comprising the field values may be followed by the serialized data for each field in the cohort and census files. The headers and/or data values may be serialized using one or more field separators (e.g., such as key?val&, key?val1, val2, . . . & . . . , and/or key?skey1:sval1, skey2:sval2, . . . & . . . ).

The cohort data and the census data may be encoded differently (e.g., with sample identifier list or sample count), as described herein. Both encoded cohort files and census files store a separate copy of value lists (e.g., val11, val12 . . . ). To serialize the metrics stored in each file, we take the order of the file name defined in the cohort header and the census header, respectively, to avoid repeating the file name in each record. The data each of the metrics are serialized together, for example, field 1 (e.g., with metric values v11, v12, v13, and bit sequence b1), field 2 (e.g., with metric values v21, bit sequence b2), field 3 (e.g., with metric values v31, v32 and bit sequence b3), etc. where the serialized data may be v11&v12&v13?b1; v21&b2; v31&v32?b3. Characters “&”, “?”, and “;” may be used to delimit the fields, since other characters may be used in the metric values in VCF files.

This serialized data of each record may be stored in a column of each row in the cohort file and census file. Each file may also include separate columns that include the chromosome name, start position, and end position of the record. These columns may be implemented by a browser extensible data (BED) format. Additional columns may be included for customized data, which may be used to store the cohort record data and census record data. Each of the rows of the cohort file and the census file may be further compressed. For example, each of the rows of the cohort and census file may be further compressed with blocks (e.g., 64 Kilo-Byte blocks) using a public bgzip algorithm. The compressed blocks may be indexed by genomic region using an indexing algorithm. An example indexing algorithm may be a public tabix algorithm.

The iterative gVCF genotyper may generate the serialized data file 1418 in the BED format with the serialized bytes of data. The BED format may be a text file format used to store genomic regions as coordinates and associated annotations. At 1420, the bit compressed and serialized cohort and census hash map data may be further encoded into ascii data such that the data (together with the region information) may be compressed using a bgzip compression algorithm and may be indexed by the tabix algorithm. The process 1400 (e.g., the bgzip compression algorithm and tabix algorithm) may allow for genomic region querying.

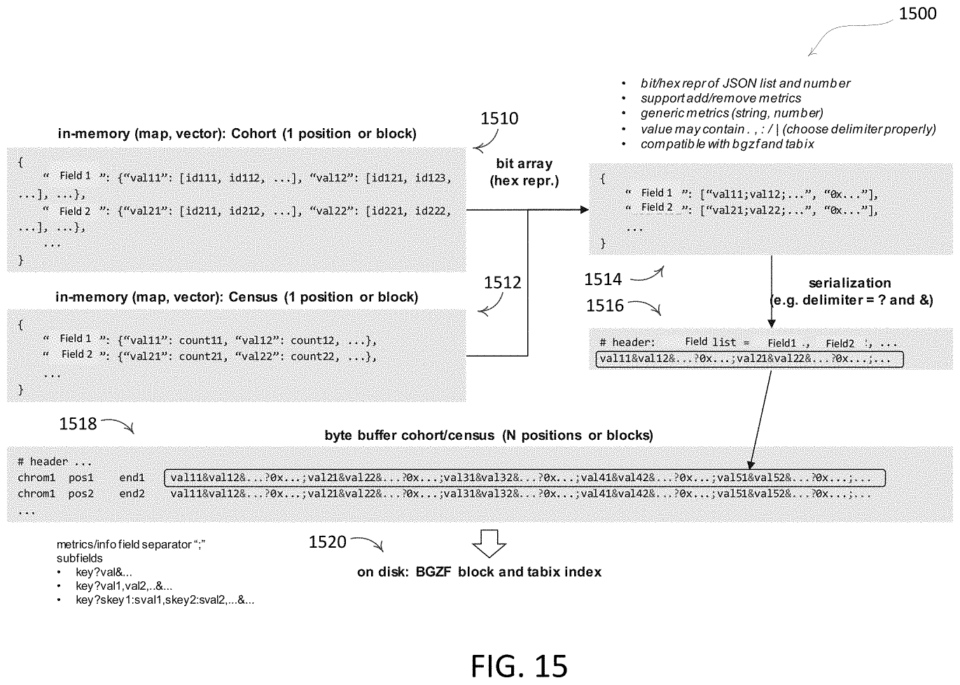

FIG. 15 depicts another example process 1500 that may be implemented by one or more computing devices (e.g., such as the server device 102, the client device 108, and/or the sequencing device 114 shown in FIG. 1) for data serialization and compression. The process 1500, or portions thereof, may be performed by an iterative gVCF genotyper executed by a processor in the one or more computing devices. In the example process 1500, cohort data and census data may be serialized into binary data using bit compression and further encoded into ascii data such that the data (together with the region information) may be compressed using the bgzip compression algorithm and may be indexed by tabix algorithm, which may allow for genomic region querying. bgzip and tabix are public algorithms implemented in HTSlib. The process 1500, or portions thereof, may be performed to more efficiently store variant data in a census file, a cohort file, and/or a msVCF file. The process 1500, or portions thereof, may be performed at a single computing device or may be distributed across multiple computing devices (e.g., multiple server(s), sequencing device(s) and/or client computing device(s)). The process 1500, or portions thereof, may reduce the amount of processing resources and/or storage space (e.g., memory) used by the computing device(s) during a variant analysis.

In the example process 1500, a cohort and a census may be combined into a bit array (e.g., a hex representation). The example process 1500 may include bit/hex representation of JSON list and number. The example process 1500 may support adding and removing metrics. The example process 1500 may support generic metrics (string, number). The example process 1500 may support values that contain periods, commas, colons, slashes, etc. and may ensure that the delimiter is chosen properly. The example process 1500 may be compatible with BGZF (e.g., BGZF block) and tabix (e.g., tabix index).

The iterative gVCF genotyper may receive cohort data 1510 in one or more cohort files. The iterative gVCF genotyper may receive census data 1512 in one or more census files and/or a global census file. The cohort data and/or census data may be separated by position or block in the cohort and/or census files. Bit compression may be performed at 1514 on the cohort and census data. For example, the cohort and census data may be represented (e.g., using the process 1200 shown in FIG. 12) using a bit array (e.g., a binary bit array). One or more headers and/or alphanumeric values in the cohort or census file headers may be serialized at 1516. The headers and/or alphanumeric values may be serialized using on e or more field separators (e.g., such as key?val&, key?val1, val2, . . . & . . . , and/or key?skey1:sval1, skey2:sval2, . . . & . . . ). At 1518, the byte buffer may be generated. The byte buffer may include cohort and/or census data. At 1520, the bit compressed and serialized cohort and census data may be further encoded into ascii data such that the data (together with the region information) may be compressed using bgzip compression algorithm and can be indexed by tabix algorithm. The process 1500 (e.g., the bgzip compression algorithm and tabix algorithm) may allow for genomic region query.

In addition to, or in alternative to, performing data compression and other memory preservation techniques described herein, the data in the files described herein may be stored in memory in a manner that preserves memory locations for overlapping data. For example, variant data in cohort files and census files may include overlapping data. The overlapping data may refer to the overlap of genomic regions (e.g., overlapping variants and/or hom-ref blocks). In one example, a first record for chr20:1000-2000 and a second record for chr20:1500-2500 may have an overlap in chr20:1500-2000. When these records are aggregated, a third record may be generated for 3 regions: chr20:1000-1499, chr20:1500-2000, chr:2001-2500. The cohort data or the census data in the first and third region may be copied from the original first record and the original second record, while the data from the second region may be updated from both the first record and the second record. The variant data may be received in a genomic variant call file comprising a record representing a genomic region. The variant data may be stored in memory locations improve storage of the variant data by leveraging the overlapping regions of different records.

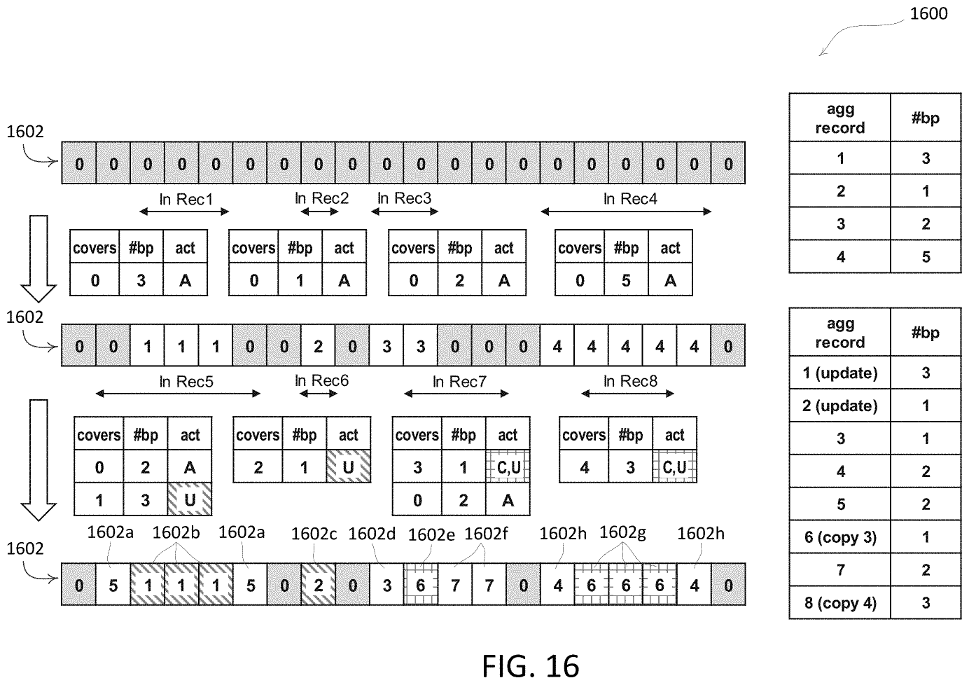

FIG. 16 depicts an example process 1600 that may be implemented by one or more computing devices (e.g., such as the server device 102, the client device 108, and/or the sequencing device 114 shown in FIG. 1) for adding, copying, and/or updating records utilizing buffer aggregation with overlapping genomic regions. For example, the example process 1600, or portions thereof, may be performed by an iterative gVCF genotyper executed by a processor in the one or more computing devices. The method 1600 may be implemented for aggregating gVCF files into cohort files and/or aggregating census files into a global census file. The process 1600, or portions thereof, may be performed to efficiently store genome variant data in a census file, a cohort file, and/or an msVCF file.

The example process 1600 may begin when the iterative gVCF genotyper receives one or more records. The records may be received from one or more input gVCF files or one or more input census files. Each of the records may include genome variant data that represent different genomic regions.

A buffer 1602 may be an output buffer including a fixed number of buffer positions (bps) for storing data in the records in each of the available buffer positions. The iterative gVCF genotyper may receive a first set of records comprising genome variant data for being stored in the buffer 1602. The first set of records may include Record 1, Record 2, Record 3, and Record 4. Each record may indicate a number of buffer positions that may be utilized to store a genomic region in the genome variant data. In the example shown in FIG. 16, the genome variant data of Record 1 may occupy three buffer positions, the genome variant data of Record 2 may occupy one buffer position, the genome variant data of Record 3 may occupy two buffer positions, and the genome variant data of Record 4 may occupy five buffer positions.

The iterative gVCF genotyper may analyze each of the records that have been received (e.g., Records 1-4) and analyze the buffer 1602 to determine the act to be performed on each record for enabling storage in the buffer 1602. For example, the iterative gVCF genotyper may analyze a received record and the buffer 1602 to determine whether there are any genomic regions in the received record and that overlap with previously received genome variant data that has been stored in the buffer 1602. If the iterative gVCF genotyper determines that the genomic regions in the record, or in one or more portions of the record, fail to overlap with the data currently stored in the buffer 1602, the iterative gVCF genotyper may perform the act of adding the record, or those portions that fail to overlap with previously stored data, to the buffer (e.g., indicated by the act “A” in each record). Here, as each of the buffer positions in the buffer 1602 is open and fails to have any data stored therein, there are no previously stored records with which any of the Records 1-4, or portions thereof, may overlap and the iterative gVCF genotyper may add each of the Records 1-4 to the buffer 1602. The iterative gVCF genotyper may store the genome variant data for each of the Records 1-4 in the buffer 1602 with at least one empty buffer position between non-overlapping adjacent records. The record may be separated with empty base pairs (e.g., genomic positions) when there is not overlapping data. After storing the Records 1-4 in the buffer 1602, the iterative gVCF genotyper may receive a second set of records for being stored in the buffer 1602. The second set of records may include Record 5, Record 6, Record 7, and Record 8. Again, each record indicates a number of buffer positions that may be utilized to store a genomic region in the genome variant data. The first batch of records may be separated with empty base pairs (e.g., genomic positions), as shown in FIG. 16, because the first batch of records does not have overlapping data, as shown in the range of Record 1, Record 2, Record 3 and Record 4 below the buffer 1602. If there were overlapping in some positions of the first batch of records, the records may be stored similarly to the overlap shown in Records 5-8.

The iterative gVCF genotyper may analyze each of the records that have been received (e.g., Records 5-8) and analyze the buffer 1602 to determine the act to be performed on each record for enabling storage in the buffer 1602. If the genomic region in a portion of the record fails to overlap with a record previously stored in the buffer 1602, the iterative gVCF genotyper may add the genome variant data comprising the genomic region to the buffer 1602 in respective buffer positions. If a genomic region in at least one portion of the genome variant data in a record overlaps entirely with the genomic regions of genomic data in a previously stored record in the buffer 1602, the overlapping buffer positions in the buffer 1602 may be updated to include the overlapping portions of the updated record. If a genomic region in at least one portion of the genome variant data in a record overlaps partially with the genomic regions of the genomic data in a previously stored record in the buffer 1602, the overlapping buffer positions in the buffer may be updated and the non-overlapping portions of the record may be copied and stored with the overlapping portions to maintain the genome variant data stored in the previous record.

For example, as shown in FIG. 6, Record 5 may include a portion of the genome variant data that may occupy three buffer positions overlaps with the entire previously stored Record 1. The portion of the genome variant data in Record 5 may include genomic regions that overlap with the genomic regions in the genome variant data of Record 1. As such, the iterative gVCF genotyper may identify that genomic regions in the genome variant data of Record 5 overlap entirely with the genomic regions previously stored for Record 1 at the buffer positions 1602b and the buffer positions 1602b may be updated with the data in Record 5 (e.g., indicated by the act “U” in the record). As Record 5 includes additional portions of the genome variant data comprising genomic regions that fail to overlap with other genome variant data in the buffer 1602, the genome variant data may be added to the buffer positions 1602a, which may be consecutive buffer positions with the updated buffer positions 1602b. For example, one or more of the updated buffer positions 1602b may be adjacent to the buffer positions 1602a. Processing Record 6, the iterative gVCF genotyper may also identify that the genome variant data in Record 6 overlaps entirely with the genome variant data previously stored for Record 2 at the buffer position 1602c and the buffer position 1602c may be updated with the data in Record 6.

The iterative gVCF genotyper may process Record 7 and identify that a portion of the genome variant data in Record 7 overlaps with a portion of the genome variant data in the previously stored Record 3. Since a portion of the previously stored Record 3 will be maintained, the iterative gVCF genotyper may copy a portion of the genome variant data for Record 3 and update a portion of the genome variant data for Record 3 (e.g., indicated by the act “C, U” in the record). The genome variant data previously stored for Record 3 at the buffer position 1602e may be updated with the data in Record 7. The non-overlapping portion of the of the previously stored Record 3 may be copied and maintained in buffer position 1602d, which is a consecutive buffer position with the buffer position 1602e comprising the overlapping genome variant data for the two records. As Record 7 also comprises a non-overlapping portion of the record, the non-overlapping portions of Record 7 may be added at buffer positions 1602f.

The iterative gVCF genotyper may process Record 8 and identify that the genome variant data in Record 8 overlaps with a portion of the genome variant data in the previously stored Record 4. Since a portion of the previously stored Record 4 will be maintained, the iterative gVCF genotyper may copy a portion of the genome variant data for Record 4 and update a portion of the genome variant data for Record 4 (e.g., indicated by the act “C, U” in the record). The genome variant data previously stored for Record 4 at the buffer positions 1602g may be updated with the genome variant data in Record 7. The non-overlapping portions of the of the previously stored Record 4 may be copied and maintained in buffer position 1602h, which may be consecutive buffer position with the buffer position 1602g comprising the overlapping genome variant data for the two records. It will be appreciated that although the example process 1600 shown in FIG. 16 uses an output buffer having 20 buffer places, the output buffer is not limited to having 20 buffer places. Instead, the output buffer could have any number of buffer places

Although features, elements, and functions are described above in particular combinations, a feature, element, or function is used alone or in any combination with the other features, elements, or functions. Various presently unforeseen or unanticipated alternatives, modifications, variations, or improvements may be subsequently made that are also intended to be encompassed by the following claims.

The methods described herein are implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random-access memory (RAM), removable disks, and optical media such as CD-ROM disks, and digital versatile disks (DVDs).

What is claimed is:

1. A computer-implemented method of iterative gVCF genotyping, the method comprising:
receiving a first plurality of genomic variant call files associated with a first batch of sequencing data;
generating a first cohort file for the first batch by aggregating data from a subset of fields in each of the first plurality of genomic variant call files;
generating a first census file that comprises variant summary statistics and hom-ref blocks of the first batch;
receiving a second plurality of genomic variant call files associated with a second batch of sequencing data;
generating a second cohort file for the second batch by aggregating data from the subset of fields in each of the second plurality of genomic variant call files;
generating a second census file that comprises variant summary statistics and hom-ref blocks of the second batch;
generating a global census file by aggregating the first census file and the second census file, wherein the global census file comprises census data from batches of samples received from sequencing devices at different sites;
generating a first multi-sample variant call file for the first batch using the first cohort file, the first census file, and the global census file; and
generating a second multi-sample variant call file for the second batch using the second cohort file, the second census file, and the global census file.

2. The computer-implemented method of claim 1, further comprising:
performing a genome-wide sequencing analysis using one or more of the first multi-sample variant call file or the second multi-sample variant call file.

3. The computer-implemented method of claim 1, wherein the first plurality of genomic variant call files associated with the first batch are split into shards of equal size, and wherein each shard is processed using one of a plurality of computation nodes.

4. The computer-implemented method of claim 1, further comprising:
receiving a third plurality of genomic variant call files associated with a third batch of sequencing data;
generating a third cohort file for the third batch by aggregating data from the subset of fields in each of the third plurality of genomic variant call files;
generating a third census file that comprises variant summary statistics and hom-ref blocks of the third batch;
updating the global census file by aggregating the third census file with the global census file; and
generating a third multi-sample variant call file for the third batch using the third cohort file, the third census file, and the updated global census file.

5. The computer-implemented method of claim 1, wherein the method is performed on a local computing system or is distributed across a cloud-computing system.

6. A system, comprising:
at least one processor; and
a computer readable medium comprising instructions that, when executed by the at least one processor, cause the at least one processor to:
receive one or more genomic variant call files associated with one or more samples;
generate, from the one or more genomic variant call files, one or more cohort files and one or more census files;
aggregate the one or more census files into a global census file, wherein the global census file comprises census data from batches of samples received from sequencing devices at different sites;
generate, based on the global census file, the one or more cohort files, and the one or more census files, at least one multi-sample variant call file; and
store the multi-sample variant call file in the memory.

7. The system of claim 6, wherein the samples comprise samples from a sequencing run, a sequencing cycle, or multiple sequencing runs.

8. The system of claim 6, wherein the instructions are further configured to cause the at least one processor to perform parallel processing using multiple compute nodes.

9. The system of claim 8, wherein the instructions, when executed by the at least one processor, further cause the processor to:
perform parallelization and multithreading by regions of sequence data.

10. The system of claim 9, wherein at least two compute nodes perform at least two levels of parallelization for processing, aggregating, or generating data for a corresponding region of the sequence data, wherein each compute node processes a specific region.

11. The system of claim 6, wherein the cohort files and the census files in a region are bit compressed and serialized.

12. A system, comprising:
at least one processor; and
a computer readable medium comprising instructions that, when executed by the at least one processor, cause the at least one processor to:
receive a plurality of genomic variant call files, each of the genomic variant call files associated with a respective sample of a plurality of samples;
identify, using reference alternate genotype (RAGT) statistics in the plurality of genomic variant call files, a plurality of reference alleles and a plurality of alternate alleles associated with the plurality of samples;
count the instances of each of the plurality of reference alleles and each of the plurality of alternate alleles;
select a normalized reference allele from the plurality of reference alleles, wherein the longest reference allele is selected as the normalized reference allele;
normalize the other reference alleles of the plurality of reference alleles by extending the other reference alleles to correspond to the normalized reference allele;
normalize the plurality of alternate alleles by extending each alternate allele the same amount that the respective corresponding reference allele was extended; and
generate a multi-sample variant call file using the normalized reference alleles and the normalized alternate alleles.

13. The system of claim 12, wherein the instructions further cause the at least one processor to generate a normalized representation of each sample using the normalized reference allele such that each of the plurality of alternate alleles are indexed using the normalized reference allele.

14. The system of claim 12, wherein the other reference alleles are extended by adding a respective number of bases to correspond to the normalized reference allele.

15. The system of claim 12, wherein the instructions further cause the at least one processor to:
receive an additional genomic variant call file associated with an additional sample;
identify a reference allele and one or more alternate alleles associated with the additional sample; and
update the normalized representation to include the reference allele and the one or more alternate alleles associated with the additional sample.

16. The system of claim 15, wherein the instructions being configured to cause the at least one processor to update the normalized representation further comprises the instructions being configured to cause the at least one processor to:
determine that the length of the reference allele is shorter than the normalized reference allele;
extend the reference allele and the one or more alternate alleles to correspond to the normalized reference allele; and
reorder the plurality of reference alleles and the plurality of alternate alleles to include the extended reference allele and the one or more extended alternate alleles.

17. The system of claim 15, wherein the instructions being configured to cause the at least one processor to update the normalized representation further comprises the instructions being configured to cause the at least one processor to:
determine that the length of the reference allele is longer than the normalized reference allele;
select the reference allele as an updated normalized reference allele;
normalize the plurality of reference alleles and the plurality of alternate alleles by extending to correspond to a length of the updated normalized reference allele; and
reorder the plurality of reference alleles and the plurality of alternate alleles to include the updated normalized reference allele and the one or more extended alternate alleles.

18. The system of claim 12, wherein the instructions further cause the at least one processor to reorder the genotype of each sample based on the normalized reference allele and the normalized representations.

19. The system of claim 18, wherein the instructions further cause the at least one processor to generate a mapping for each of the plurality of alternate alleles based on the normalized reference allele.

20. The system of claim 19, wherein the mapping for each of the plurality of alternate alleles is stored in site information in a census file.

21-34. (canceled)
